(19) United States
(12) Patent Application Publication
(10) Pub. No.: US 2024/0398894 A1
(43) Pub. Date: Dec. 5, 2024

(54) ANGIOGENIC CONTROL, PREFERABLY COMBINED WITH GLYCAEMIC CONTROL

(71) Applicants: BIOTEMPT B.V., Den Haag (NL); RESILIUN B.V, Koekange (NL)

(72) Inventor: Gert WENSVOORT, Koekange (NL)

(73) Assignees: BIOTEMPT B.V., Den Haag (NL); RESILIUN B.V., Koekange (NL)

(21) Appl. No.: 18/698,512

(22) PCT Filed: Oct. 5, 2022
(86) PCT No.: PCT/NL2022/050558

§ 371 Date: Apr. 4, 2024

Related U.S. Application Data
(60) Provisional application No. 63/252,488, filed on Oct. 5, 2021.

(51) Int. Cl. A61K 38/17; A61K 38/28

(52) U.S. Cl. CPC A61K 38/1709; A61K 38/28

(57) ABSTRACT

The invention relates to a distinct and newly emerging class of drugs: peptide modulators of mTOR that act as autophagy inhibiting compounds and target the nutrient sensing system of the mechanistic target of rapamycine (mTOR) and induce angiogenic activity. The invention provides formulations, methods, and means to prevent or treat vasculopathy, in particular diabetic vasculopathy, to achieve or maintain angiogenic control, preferably concomitant with glycaemic control.

### FIELD OF THE INVENTION

The invention relates to a distinct and newly emerging class of drugs: peptide modulators of mTOR that act as autophagy inhibiting compounds and target the nutrient sensing system of the mechanistic target of rapamycine (mTOR) and inhibit autophagy. The invention provides pharmaceutical formulations, methods, and means to exert angiogenic control, for solitary uses, and in particular combined with established insulin formulations, methods, and means to exert glycaemic control concomitant with said angiogenic control.

### BACKGROUND

Diabetes mellitus is an endocrine disorder due to an overall deficiency of insulin or defective insulin function which causes hyperglycaemia. Type 1 diabetes is usually seen in younger patients and accounts for 5% to 10% of cases worldwide and is thought secondary to autoimmune destruction of B-islet cells of the pancreas. Type 2 diabetes accounts for 90% to 95% of cases worldwide and is thought due to genetic and environmental factors with resultant insulin resistance and pancreatic beta-cell dysfunction. Complications arising from hyperglycemia can either be macrovascular or microvascular. The macrovascular disease affects mainly the cardiovascular and cerebrovascular systems, and the microvascular disease includes nephropathy, retinopathy, and neuropathy. Glycaemic control in diabetes mellitus often entails replacing the actions of the beta cells of the pancreatic islet to detect the needs of insulin and to have insulin administered according to the needs of the patient's body. Insulin is a natural hormone, and it is an essential medication for a multitude of disease states. One of the most critical uses of insulin is in type 1 diabetes mellitus and type 2 diabetes mellitus. Insulin is one of the few medications indicated for use in the management of gestational diabetes. In patients with diabetes mellitus (Dave H D, Preuss C V. Human Insulin. [Updated 2021 Feb. 17]. In: StatPearls [Internet]. Treasure Island (FL): StatPearls Publishing; 2021 January-. Available from: https://www.ncbi.nlm.nih.gov/books/NBK545190/), to reach the goal of normal glycemic control with similar 24-hour insulin activity like in healthy adults without diabetes mellitus, one single insulin formulation with a defined onset, peak, and duration of action is often considered not helpful. Hence there is a need for different kinds of insulin that have different pharmacokinetics. Based on the mode of action, there are at least four different types of insulin analogues. The rapid-acting insulin has a rapid onset of action (about less than 30 minutes), peak action at about 1 hour, and short duration of action (up to 5 hours). Insulin Lispro and Insulin Aspart are examples of rapid-acting insulins. These insulins help achieve glycaemic control, specifically in the postprandial state. The short-acting insulin analogues activity begins in about 30 to 60 minutes, peaks at 2 to 4 hours, and activity last for about 8 hours. These insulin analogues, such as insulin Glulisine must be administered approximately 20 to 30 minutes before meals for effectiveness. Rapid- or short-acting insulin analogues (such as insulin Lispro, insulin Aspart and insulin Glulisine) typically act more quickly than regular human insulin. The intermediate-acting insulin analogues have an onset of activity at around 1 to 2 hours, peak action at 6 to 10 hours, and a duration of action up-to about 16 hours. Neutral protamine Hagedorn (NPH) and Lente insulin are examples of intermediate-acting insulin analogues. Some of the long-acting insulin analogues are insulin Detemir and insulin Glargine. Their activity begins at around 2 hours, peak effect from 6 to 20 hours, and lasts up to about 36 hours. Intermediate- and long-acting (basal) insulins are recommended for patients with type 1, type 2, or gestational diabetes. They may also be used in other types of diabetes (i.e. steroid-induced). Persons with type 1 diabetes generally use intermediate-acting insulin or long-acting insulin in conjunction with regular or rapid-acting insulin to achieve glycaemic control. Persons with type 2 diabetes may use intermediate or long-acting insulins in conjunction with regular or rapid-acting insulins or with oral medications to achieve glycaemic control.

However, achieving or exerting glycaemic control does not resolve all ailments of patients with endogenous insulin deficiencies. In particular, diabetic vasculopathies, such as retinopathy, peripheral neuropathy (DPN) in overall conditions with deficiency of insulin are well-known microvascular complication of diabetes mellitus that do not resolve even with the best of glycaemic control. DPN, for example, leads to further infections and increases the risk of diabetic foot ulcers (FIG. 1), non-traumatic amputations and mortality. Although hyperglycaemia as well as hypoglycaemia plays an important role in the development of DPN, intensive glycaemic control (with or without insulin) does not eliminate the risk of developing DPN in patients with diabetes, suggesting that other factors than lack of insulin and/or instable glucose metabolism may be involved in DPN development. C-peptide levels in the peripheral blood are widely accepted as the most appropriate measure of insulin secretion, and are not eliminated in the first-pass metabolism through the liver. Previously considered to be an inactive by-product of insulin synthesis, C-peptide is a hormonally active peptide. In type 1 diabetes, an Italian study (Panero et al. Fasting plasma C-peptide and micro- and macrovascular complications in a large clinic-based cohort of type 1 diabetic patients. Diabetes Care. 2009; 32:301-5) demonstrated a significant and negative association between C-peptide levels and microvascular complications, including neuropathy. Typically C-peptide deficiency associates with these microvascular complications, incentivizing attempts to treat such patients with exogenous C-peptide. Several studies in animal models of diabetes and in patients with type 1 diabetes have indeed demonstrated beneficial effects of C-peptide replacement on both peripheral and autonomic nerve function in diabetes (Johansson et al. Beneficial effects of C-peptide on incipient nephropathy and neuropathy in patients with Type 1 diabetes mellitus. Diabetic Med. 2000; 17:181-9.) In type 2 diabetes, a Chinees study (Qiao et al. C-peptide is independent associated with diabetic peripheral neuropathy: a community-based study. Diabetol Metab Syndr 9, 12 (2017)), C-peptide levels were significantly and negatively associated microvascular complications with DPN as well. It is thought that endogenous C-peptide contributes to resolve of vasculopathy through angiogenic control of our vascular system, exerting angiogenic activities on said vascular system. In patients with low C-peptide levels, a high prevalence of vasculopathy with retinopathy, nephropathy and neuropathy is observed. Peripheral neuropathic changes to the body result in decreased pedal sensation and make the diabetic foot prone to wounds from mechanical or pressure injuries. Microvascular changes can result in reduced blood flow to the extremities and delaying healing of wounds. This complication may be prevented, as the inciting factor is most often minor trauma. Early identification of these cutaneous injuries also can lead to improved outcomes while decreasing the risk of progression.

However, a clinical trial evaluating the efficacy and safety of a long-acting (PEGylated) C-peptide in subjects with type 1 diabetes and neuropathy failed to meet its primary endpoint (Wahren et al., Long-acting C-peptide and neuropathy in type 1 diabetes: A 12-month clinical trial. Diabetes Care. 2016 April; 39 (4): 596-602.) A total of 250 patients with type 1 diabetes and peripheral neuropathy received long-acting (PEGylated) C-peptide in weekly dosages of 0.8 mg (n=71) or 2.4 mg (n=73) or placebo (n=106) for 52 weeks. Once-weekly subcutaneous administration of long-acting C-peptide for 52 weeks did not improve bilateral sural nerve conduction velocity (SNCV), other electrophysiological variables, or the modified Toronto Clinical Neuropathy Score (mTCNS), but only resulted in improvement of vibration perception threshold (VPT) compared with placebo, C-peptide in this formulation bringing no angiogenic control.

Infectious gangrene of the foot is a serious complication of diabetes. It usually leads to a certain level of lower-extremity amputation (LEA). Foot gangrene is defined as dead tissue in the foot resulting from inadequate blood flow supply. It is one of the final manifestations of critical limb ischemia. It can be caused by obstructed peripheral circulation or microbial infections. Foot ulcers in patients with diabetes are at an increased risk of initiating foot gangrene, mainly due to peripheral arterial disease. Foot gangrene ranges from two extremes: (FIG. 1A) from early manifestation of foot ulcers with developing dry gangrene with ischemic and necrotic tissue but no infection to (FIG. 1B) late manifestation of wet gangrene with both infectious and necrotic tissue. Dry gangrene can thus lead up to wet gangrene. In the more pronounced stages of dry gangrene, the affected area is often characterized by a dark green or purple, almost black colour. Wet gangrene has a wet appearance, with blisters and swelling. Wet gangrene may also occur in people who have frostbite or experience a severe burn. People with diabetes may ultimately develop wet gangrene after experiencing a minor toe or foot injury, for example such as seen in athlete's foot. Blood flow to the extremities is generally diminished in people with diabetes. This means that the tissue in these areas is unable to heal quickly. As a result, infection can develop more easily. Wet gangrene can spread quickly and, if left untreated, can be fatal. Typically, such patients rapidly develop sepsis and die.

Treatment for all forms of gangrene involves removing dead tissue, treating and stopping a possible spread of infection, and treating the condition that caused the gangrene. Treatment may include surgery (also called debridement) to remove dead tissue to keep an infection from spreading. It may be necessary to remove an affected limb, hand, finger, foot or toe. Sometimes, instead of surgery, maggots from fly larvae are placed on the ulcer wound where they eat the dead and infected tissue without hurting healthy tissue. Antibiotics may be used to treat or prevent infection and hyperbaric oxygen therapy may treat wet gangrene or ulcers related to diabetes or peripheral artery disease.

Infectious gangrene is thus both a limb- and life-threatening disease. Proper medical treatment with antibiotics and wound dressing may not be effective without timely surgery to remove the necrotic and infectious tissue, thus ultimately leading to limb amputation. However, reports have shown that patients with diabetes-related amputations still have a high risk of mortality, with a 5-year survival rate of 40-48% regardless of the aetiology of the amputation (Huang et al., Survival and associated risk factors in patients with diabetes and amputations caused by infectious foot gangrene. J Foot Ankle Res. 2018 Jan. 4; 11:1), see also FIG. 2.

Patients with diabetes mellitus (type 1 or 2) have a total lifetime risk of a diabetic foot ulcer complication as high as 25% (some report 50%), in England alone already with an estimated cost of £935 million to the NHS. (Diabetic foot care in England: an economic study-Marion Kerr, Insight Health Economics, 2017). The end result of ill-treated diabetic ulcers is increased overall morbidity (Packer C F, Ali S A, Manna B. Diabetic Ulcer. [Updated 2021 Feb. 20]. In: StatPearls [Internet]. Treasure Island (FL): StatPearls Publishing; 2021 January-. Available from: https://www.ncbi.nlm.nih.gov/books/NBK499887/).

As indicated, diabetic ulcer formation is aggravated and sped up under conditions of deteriorated glycaemic control. Peled et al., (Association of Inpatient Glucose Measurements With Amputations in Patients Hospitalized With Acute Diabetic Foot, The Journal of Clinical Endocrinology & Metabolism, Volume 104, Issue 11, November 2019, Pages 5445-5452) find that patients experiencing any hyperglycaemia and any or severe hypoglycaemia were more likely to undergo any or major amputations during hospitalization. High glycaemic variability was associated with major amputations. Peripheral vascular disease (PVD), high Wagner score, and hypoglycaemia were independent predictors of amputations. Older age, peripheral vascular disease (PVD), previous amputation, elevated white blood cell level, high Wagner score, and hypoglycaemia were independent predictors of major amputations. In a systematic review of 60 observational studies, of which 47 were included in a meta-analysis (Lane et al., Glycemic control and diabetic foot ulcer outcomes: A systematic review and meta-analysis of observational studies. J Diabetes Complications. 2020 October; 34 (10): 107638), hyperglycemia (higher A1C and higher fasting glucose) was associated with increased likelihood of lower extremity amputation among subjects with diabetic foot ulcers. Lane et al's findings suggest that A1C levels of 8% or greater and fasting glucose levels of 126 mg/dl and greater are associated with increased likelihood of lower extremity amputation in patients with existing diabetic foot ulcers.

Diabetic ulcers are not only debilitating to a patient in themselves, the risk of nontraumatic lower limb amputations because of ill-treatable foot ulcer is 15 times higher in diabetic patients when compared with nondiabetics. Patients with diabetes are at high risk for lower extremity amputations, higher healthcare costs, and lower quality of life (Song K, Chambers A R. Diabetic Foot Care. [Updated 2021 Jul. 31]. In: StatPearls [Internet]. Treasure Island (FL): StatPearls Publishing; 2021 January-. Available from: https://www.ncbi.nlm.nih.gov/books/NBK553110/). In a systematic study regarding non-traumatic amputations of patients with diabetes mellitus and peripheral vascular disease (Thorud et al., Mortality After Nontraumatic Major Amputation Among Patients With Diabetes and Peripheral Vascular Disease: A Systematic Review. J Foot Ankle Surg. 2016 May-June; 55 (3): 591-9), the 5-year mortality rate of patients after a below-the-knee amputation was 40 to 82%, and mortality after an above-the-knee amputation was 40 to 90%. Many of these complications are preventable by a thorough annual foot exam and routine foot care performed by the patient. Patient education about correct foot care practices is the current cornerstone of prevention of diabetic foot disease. Foot ulcers are a common reason for hospital stays for people with diabetes. It may take weeks or even several months for foot ulcers to heal. Diabetic ulcers are often painless (because of decreased sensation in the feet). Treatment of ulcerated diabetic foot is a complex problem (Lepäntalo et al., Chapter V: Diabetic foot. Eur J Vasc Endovasc Surg. 2011 December; 42 Suppl 2: S60-74). Ischaemia, neuropathy and infection are three pathological components that lead to diabetic foot complications, and they frequently occur together as an aetiologic triad. Neuropathy and ischemia are the initiating factors, most often together as neuroischaemia, whereas infection is mostly a consequence. Furthermore, the healing of an ulcer is hampered by microvascular dysfunction. Guidelines have largely ignored these specific demands related to ulcerated diabetic feet. Any diabetic foot ulcer should always be considered to have vascular impairment unless otherwise proven. Early referral, vascular testing, imaging and intervention are crucial to improve diabetic foot ulcer healing and to prevent amputation. Timing is essential, as the window of opportunity to heal the ulcer and save the leg is easily missed. There is however a paucity of data on the best way to diagnose and treat these diabetic patients. Most of the studies dealing with diabetic feet are not comparable in terms of patient populations, interventions or outcome.

Diabetes mellitus likely disrupts wound repair and leads to the development of chronic wounds due to impaired angiogenesis. Autonomic neuropathy afflicts some 40% of the patients with diabetes mellitus of more than 15 years duration. It may evolve through defects in thermoregulation, impotence and bladder dysfunction followed by cardiovascular reflex abnormalities. Late manifestations may include generalized sweating disorders, postural hypotension, gastrointestinal problems, and reduced glycemic control. The latter symptom has grave clinical implications that involve a myriad of comorbidities including the serious complications of poor wound healing, chronic ulceration, and resultant limb amputation. In skin wound healing, which has definite, orderly phases, diabetes leads to improper function at all stages. While the aetiology of chronic, non-healing diabetic wounds is multi-faceted, the progression to a non-healing phenotype is closely linked to poor vascular networks indicating poor vasculogenesis/angiogenesis. These terms are often used interchangeably (herein as well to denote the various aspects of vessel formation and repair), however, strictly speaking vasculogenesis is defined as the formation of new vessels from precursor cells that coalesce into primitive vascular networks, and angiogenesis is the formation of new capillaries from the established vasculature. This distinction depicts the fact that blood vessels develop via two subsequent processes, vasculogenesis and angiogenesis, both being of crucial importance for blood vessel maintenance and repair. These processes are also involved in the development of the foetal and placental vasculatures. Blood vessel development (neovessel formation, Rafii S, Lyden D. Therapeutic stem and progenitor cell transplantation for organ vascularization and regeneration. Nat Med. 2003 June; 9 (6): 702-12; Masuda, H and Asahara, T. Post-natal endothelial progenitor cells for neovascularization in tissue regeneration, Cardiovascular Research, Volume 58, Issue 2, May 2003, Pages 390-398,) is a regulated process involving the proliferation, migration, and remodelling of endothelial cells (ECs), operated by in situ proliferation and migration of pre-existing endothelial cells from adjacent pre-existing blood vessels (angiogenesis) or following differentiation of endothelial progenitor cells (EPCs) from mesodermal precursors (vasculogenesis). Vasculogenesis and angiogenesis are important and complex processes involving extensive interplay between cells and growth factors. During vasculogenesis, formation of the earliest capillaries is achieved by in situ differentiation of hemangiogenic stem cells that are derived from pluripotent mesenchymal cells. The subsequent process, angiogenesis, is characterized by development of new vessels through angiogenic activity with proliferation of cells arising from already existing vessels and vascular cells, and is a well-coordinated process initiated by stimulation of various growth factors. Typically, neovascularization in tissue regeneration and wound repair (i.e. post-natal) mainly involves angiogenic activities. Vasculogenic activities are typically studied in vitro in an colony assays (clonogenic assay or colony formation assay), an in vitro cell survival assay based on the ability of a single cell to grow into a colony. Angiogenic activities are typically studied in vitro in sprouting assays (tube formation assay or angiogenesis assay), an in vitro cell survival assay based on the ability of tested cells to form capillary-like structures in vitro, which recapitulates angiogenesis. In our body, endothelial cells are surrounded by the basement membrane, which is a thin and highly specialized extracellular matrix (ECM). When endothelial cells, such as human umbilical vein cells (HUVECs), are seeded onto a basement membrane-like surface (e.g., Matrigel®) they form branching tubes or spouts that resemble capillaries.

During the past decade, numerous studies in both humans and animals have demonstrated that C-peptide, although not influencing blood sugar control, might play a role in preventing and potentially reversing some of the chronic complications of type 1 diabetes. Lim et al., (Proinsulin C-peptide prevents impaired wound healing by activating angiogenesis in diabetes. J Invest Dermatol. 2015 January; 135 (1): 269-278) demonstrated that human C-peptide can protect against vasculopathy in diabetes and investigated the potential roles of C-peptide in protecting against impaired wound healing by inducing angiogenesis using streptozotocin-induced diabetic mice and human umbilical vein endothelial cells. Diabetes delayed wound healing in mouse skin, and C-peptide supplement using osmotic pumps significantly increased the rate of skin wound closure in diabetic mice. Furthermore, C-peptide induced endothelial cell migration and tube formation in dose-dependent manners, with maximal effect at 0.5 nM. C-peptide-enhanced angiogenesis in vivo was demonstrated by immunohistochemistry and Matrigel plug assays. Lim et al's findings highlight an angiogenic role of C-peptide and its ability to protect against impaired wound healing, which may have significant implications in reparative and therapeutic angiogenesis in diabetes, and pose that C-peptide replacement is a promising therapy for impaired angiogenesis and delayed wound healing in diabetes. A consistent lack of C-peptide may indeed result in microvascular lesions that can be remedied with exogenous C-peptide, indicating that C-peptide is involved to the good in the vascular repair process. When beta-cells are defunct and little or no C-peptide is excreted, said microvascular complications may lead to retinopathy, nephropathy, and neuropathy, such complications may possibly be remedied by treatment with C-peptide. To investigate whether C-peptide could normalize impaired wound healing in diabetic mice, C-peptide was continuously supplemented using subcutaneously implanted osmotic pumps in streptozotocin-induced diabetic mice. Wounds of 4-mm diameter were created on the dorsal surface of the hindlimbs of normal, diabetic, and C-peptide-supplemented diabetic mice. Diabetic mice exhibited a significantly slower healing rate compared with normal mice. However, C-peptide supplement accelerated wound closure in diabetic mice. This effect was quantitatively analysed by Lim et al. (ibid) measuring wound diameter. These results indicate that C-peptide significantly prevents against impaired wound healing including wound closure in diabetic mice. Because angiogenesis is a crucial process for wound healing, Lim et al., (ibid) investigated whether C-peptide could activate endothelial cell migration, proliferation, and tube formation in an in vitro assay, also called a sprouting assay. It was confirmed that C-peptide induces sprouting by activating endothelial cell migration, proliferation, and tube formation, which are essential for angiogenesis. However, these promising pre-clinical studies contrast starkly with human clinical trials with C-peptide that have as yet failed to bring any progress in controlling diabetic vasculopathy

In short, there is an urgent need for a paradigm shift in care for patients having diabetic vasculopathies such as nephropathy, retinopathy and neuropathy in patients having C-peptide deficiency (typically in type 1- and end-phase type 2-diabetes) and thus ultimately also in diabetic ulcer care; that is, a new approach and classification of diabetics with vascular impairment in regard to clinical practice and research. New strategies must be developed and implemented for diabetic vasculopathy patients, such as patients having nephropathy, retinopathy and/or neuropathy and ultimately for ulcer patients with vascular impairment, to improve healing, to speed up healing rate and to avoid amputation, irrespective of the intervention technology chosen. Focused studies on the value of predictive tests, new treatment modalities as well as selective and targeted strategies are needed. As specific data on ulcerated diabetic feet are scarce, recommendations are often of low grade. In short, beyond existing and well-tested strategies of glycaemic control, for many of these patients also some form of angiogenic control is sought.

Nutrient sensing is important to sustain normal cell growth and proliferation. mTOR complex 1 (mTORC1) senses nutrients to regulate cell growth, autophagy, and other mTORC1-mediated processes. Growth factors such as insulin, IGF, PDGF and VEGF, amino acids, energy status, and stress, control mTORC1. Amino acids are essential for mTORC1 activation. Growth factors alone cannot attain maximal mTORC1 activity without amino acid supplementation (Sancak et al., The Rag GTPases bind raptor and mediate amino acid signaling to mTORC1. Science. 2008 Jun. 13; 320 (5882): 1496-501). Increased intra-cellular amino acid concentrations promote mTORC1 lysosomal localization and subsequent activation.

On the one hand, the invention provides use of a distinct and newly emerging class of drugs: peptide modulators of mTOR that act as autophagy inhibiting compounds for use in inducing the angiogenic activities required to establish the angiogenic control to combat said vasculopathies, that even occur in those patients that are otherwise under proper glycaemic control. The invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and valine (V). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and leucine (L). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), leucine (L), and proline (P). In a preferred embodiment, the invention provides an autophagy inhibiting modulator of mTOR for use in inducing angiogenic activity in prevention or treatment of a vasculopathy of a subject, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), valine (V), leucine (L), and proline (P). In another preferred embodiment, the invention provides a modulator according to the invention for use in prevention or treatment of a nephropathy, in particular for use in prevention of end-stage renal disease. In another preferred embodiment, the invention provides a modulator according to the invention for use in prevention or treatment of a retinopathy, in particular for use in prevention of partial blindness or loss of sight. In another preferred embodiment, the invention provides a modulator according to the invention for use in prevention or treatment of a neuropathy, more preferably for use in prevention or treatment of a peripheral diabetic neuropathy, more preferably for use in prevention or treatment of an diabetic ulcer. It is in particular preferred that such an autophagy-inhibiting peptide modulator of mTOR according to the invention is used when said subject is also treated to achieve or maintain glycaemic control, especially when said subject is also treated with an insulin to achieve or maintain glycaemic control. The invention provides a method for identifying a source of, preferably L-proteinogenic, amino acids, preferably a peptide, capable of inducing angiogenic activity, comprising providing cells with a peptide comprising L-proteinogenic amino acids, said amino acids for least 50% selected from the group of alanine (in one letter code: A), glutamine (Q), glycine (G), valine (V), leucine (L), isoleucine (I), proline (P), arginine (R) and asparagine (N), and determining angiogenic activity in a sprouting assay. Angiogenic activity can be determined by assessing capillary-tube formation as well as by assessing capillary branch formation, as provided herein. It is preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50%, more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R) and asparagine (N)). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N), more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). The invention also provides a method for inducing angiogenic activity, comprising providing cells with a peptide as a source of, preferably L-proteinogenic, amino acids, said peptide consisting of amino acids that are for at least 50%, more preferably for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). It is more preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and valine (V). It is more preferred that said amino acids are for at least 50%, more preferably at least 60%, more preferably at least 80%, more preferably at least 85%, more preferably at least 90%, more preferably at least 95% selected from the group of leucine (in one letter code: L), glutamine (Q), glycine (G), and valine (V). It is moreover preferred that said amino acids are for at least 60%, preferably at least 75%, more preferably at least 80%, more preferably at least 85%, more preferably at least 90%, more preferably at least 95% selected from the group of alanine (in one letter code: A), glutamine (Q), glycine (G), and leucine (L). It is moreover preferred that said amino acids are for at least 60%, more preferably at least 80%, more preferably at least 85%, more preferably at least 90%, more preferably at least 95% selected from the group of alanine (in one letter code: A), valine (V), leucine (L), and proline (P). The invention also provides a method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P), arginine (R) and asparagine (N). The invention also provides a method of treatment according to the invention, wherein said source of, preferably L-proteinogenic, amino acids, preferably a peptide, is identifiable with a method as provided herein. The invention in particular provides a method of treatment according to the invention wherein said source or peptide has an amino acid sequence that is derived from a peptide or protein shown or suspected of having angiogenic activities. Often, in the proteome of an organism, such angiogenic sequences in proteins are located close to or between more N- and C-terminal located arginine (R) or lysine (K) residues, allowing enzymes such as convertases to cut out said angiogenic amino acid sequence so that it can be used as an autophagy inhibiting peptide modulator of mTOR. It is preferred to correlate peptide use with the species of cells it is used in. For example, it is preferred that if the peptides are used in human cells as an autophagy inhibiting peptide modulator of mTOR, said peptides derive from the human proteome. Likewise, in cells of another species, such as species X, it is preferred to use peptide that derives from the species X proteome. In a preferred embodiment, the invention provides said autophagy-inhibiting peptide which has an amino acid sequence that is derived from a chorionic gonadotropin (CG), a protein involved in angiogenic activities in pregnancies, preferably said peptide is derived from a beta-chain of CG, preferably from a loop 2 of said beta-chain. Human CG (hCG) has an angiogenic amino acid sequence MTRVLOGVLPALPQVVCNYR wherein the core angiogenic sequence is located between N- and C-terminal located arginine (R) residues. Furthermore, this angiogenic core comprises an ERC-binding motif xGxxPx. Splitting up this motif facilitates use of hCG derivatives LQGV, VLPALP AQGV, LAGV, LQAV, LOGA, ALPALP, VAPALP, VLAALP, VLPAAP, and VLPALA as autophagy inhibiting peptide modulators in combination with rapid- and short-acting insulin preparations, in particular to exert both glycaemic as well as angiogenic control. Peptide VLQGVLPALPQVV, for example, finds a better use in combination with intermediate- and long-acting insulin preparations where it is released more slowly and more in line with the release rates of the insulins. In another preferred embodiment, the invention provides said autophagy-inhibiting peptide wherein said peptide has an angiogenic amino acid sequence that is derived from a C-peptide. In the pre-pro-insulin molecule human C-peptide has sequence RREAEDLQVGQVELGGGPGAGSLQPLALEGSLQKR, indeed the angiogenic core sequence is flanked by arginine (R) or lysine (K) residues. Furthermore, this angiogenic core comprises an ERC-binding motif xGxxPG. As ERC-binding motifs are involved in coacervation trough oligomerization of peptides having these sequences, such autophagy inhibiting peptides having an ERG-binding motif typically have a slower release rate when injected and are typically more useful and preferred under circumstances wherein such slow-release is desired, such as in combination formulations with intermediate- or, more preferably, long-acting insulins. Coacervation typically involves aggregation of colloidal droplets of the peptides held together by electrostatic attractive forces, which explains the slow-release nature of such ERC-motif comprising peptides when injected, at least in comparison with peptides without ERC-motif and not showing coacervation, that typically have faster release characteristics. Preferred C-peptide fragment peptides for use as autophagy inhibiting peptide modulator of mTOR in treatment of angiogenic dysfunction, in particular in circumstances of C-peptide deficiency are preferably isolated and/or synthetic, preferably non-peggylated, C-peptide fragments EAEDLQVGQVELGGGPGAGSLQPL, DLQVGQVELGGGPGAGSLQP, LVGQVELGGGPGAGSLQPL, QVGQVELGGGPGAGSLQPL, ELGGGPGAGSLQPL, DLQVGQVELGGGPGAGSLQPLALEGSLQ, GQVELGGGPGAGSLQPLALEGSLQ, and LGGGPGAGSLQPLALEGSLQ, LVGQVELGGGPGAGSLQPL, LGGGPGAGSLQPL, and LQVGQVELGGGPG, and functional equivalents all comprising an xGxxPG motif are thus most suitable and preferred for inclusion in or combination with an intermediate- or long-acting insulin. LQVGQVELGG and/or GGPGAGSLQPL and functional equivalents not comprising an xGxxPG motif are thus most suitable and preferred for inclusion in or combination with an rapid- or short-acting insulin.

In another preferred embodiment, the invention provides said autophagy-inhibiting peptide wherein said peptide has an angiogenic amino acid sequence that is derived from a galectin-3. The mature N-terminal fragment of human galectin-3 PQGWPGAWGNQPAGAGGYPGASYPGAYPGQAPPGAYPGQAPPGAYPGAPGAYPGAPAPGVYPGP PSGPGAYPSSGQPSATGAYPATGPYGAPAGPLIVPYNLPLPGGVVPRM, is typically characterized by multiple ERC-binding motifs xGxxPG, angiogenic core sequences are also liberated by hydrolyses. Typically (Pineda et al., Trypanosoma cruzi cleaves galectin-3 N-terminal domain to suppress its innate microbicidal activity. Clin Exp Immunol. 2020 February; 199 (2): 216-229), human pathogen T. cruzi not only binds, but also hydrolyses human N-terminal galectin-3. Remarkably, this mechanism prevents galectin-3-mediated parasite death, suggesting that T. cruzi may have developed complex strategies to modulate galectin-3 functions to successfully infect, survive and thrive within its mammalian hosts. In fact, non-pathogenic T. rangeli binds galectin-3 but does not modify protein structure. Thus T. cruzi cleaves the N-terminal collagen-like domain, rendering the C-terminal galectin-3 unable to oligomerize via coacervation, and allowing N-terminal fragments with motif xGxxPG to interact with the ERC and modulate mTOR to the good of the parasite. Specifically, the N-terminal sequences of 3 fragments obtained through parasite hydrolyses were: band 1: AGGYPGASYPG, band 2: GAPGAYPGAP and band 3: GAPAGPLIVP, indicating mTOR modulating characteristics of galectin-3 N-terminal peptide fragments derived from AGGYPGASYPGAYPGQAPPGAYPGQAPPGAYP, GAPGAYPGAPAPGVYPGPPSGPGAYPSSGQPSATGAYPATGPY and GAPAGPLIVPYNLPLPGGVVP in man. For example, fragments GAYPGAPGAYPGAPAPGV and PGAYPG have angiogenic core activities, as demonstrated herein, making PGAYPGQAPPGAYPG, PGAYPGQA and GQAPPGAYPG also useful autophagy inhibiting peptides for use in man, in particular when included in or with intermediate- or long-acting insulins. The invention also provides a method of treatment and use therein according to the invention wherein said peptide has an amino acid sequence that is derived from a lutropin (LH), a protein often monthly involved in angiogenic activities in women, preferably said peptide is derived from a beta-chain of LH, preferably from a loop 2 of said beta-chain. Beta-2-loop of human LH (hLH) has an angiogenic amino acid sequence MMRVLQAVLPPLPQVVCTYR wherein the core angiogenic sequence is located between N- and C-terminal located arginine (R) residues. Furthermore, this angiogenic core does not comprise an ERC-binding motif xGxxPx, facilitating use of LH-derived sequences, such as VLQAVLPPLPQVV, VLQAVLP, VLQA, LQAVLP, LOAV, PLPQVV, and PPLQV, and further derivatives as mTOR modulator in rapid- and short-acting insulin formulations. The invention also provides a method of treatment and use therein according to the invention wherein said peptide has an amino acid sequence that is derived from an extra-cellular matrix protein (ECM). ECM proteins comprised an abundant source of autophagy inhibiting amino acids and are useful as templates for design of autophagy-inhibiting peptides. Proline as substrate is stored in elastin and collagen in extracellular matrix, connective tissue, and bone and it is rapidly released from this reservoir by the sequential action of matrix metalloproteinases, peptidases, elastases and prolidase. As the only proteinogenic secondary amino acid, proline has special biological effects, serves as a regulator of all protein-protein interactions and responses to metabolic stress, and initiates a variety of downstream metabolic activities, including autophagy (Kadowaki et al. Nutrient control of macroautophagy in mammalian cells. Mol Aspects Med. 2006 October-December; 27 (5-6): 426-43). The invention also provides a method of treatment and use therein according to the invention wherein said extra-cellular matrix protein is an elastin. Typical core-angiogenic activities have been demonstrated with peptides VGVAPG and VGVAPGVGVAPGVGVAPG herein, both derived from the exon 24 region of human elastin. The invention also provides a method of treatment and use therein according to the invention wherein said extra-cellular matrix protein is a collagen, Collagen is rife with PGP sequences that may be useful. Typical for some collagens (here examples from human COL6A5 are shown) that comprise angiogenic core peptides flanked by R or K, such as RRAQGVPQIAVLVTHR, identifying core angiogenic sequence AQGVPQIAVLV, and derivatives such as AQGVPQ, AQGVPQI, AQGVPQIA, GVPQIAVLV, PQIAVLV, IAVLV, and others. More of such sequences may be found in collagen stretches such as (in human COL6A5) RGAPGQYGEKGFPGDPGNPGQNNNIKGQKGSKGEQGRQGRSGQKGVQGSPSSRGSRGREGQRGLR GVSGEPGNPGPTGTLGAEGLQGPQGSQGNPGRKGEKGSQGQKGPQGSPGLMGAKGSTGRPGLLGKK GEPGLPGDLGPVGQTGQRGRQGDSGIPGYGQMGRKGVKGPRGFPGDAGQK, that are found to demonstrate repeat occurrences of angiogenic core peptide sequences flanked by R or K repeats and from which desirable peptide modulators of mTOR may be selected. Another angiogenic protein of which a peptide is provided herein is human alpha-fetoprotein wherein a peptide with sequence KDLCQAQGVALQTMK is observed, comprising an antigenic core QAQGVALQ, and derivatives AQGV, AQGVA, AQGVAL, QGVALQ and GVALQ are found, all useful as autophagy inhibiting peptide modulator of mTOR, preferably in combination with rapid- or short-acting insulins. Typically, autophagy-inhibition by dipeptide modulator of mTOR AQ has recently been demonstrated in piglets, having improved modulating characteristics over amino acids A+Q (Zhang et al., Alanyl-glutamine supplementation regulates mTOR and ubiquitin proteasome proteolysis signaling pathways in piglets. Nutrition. 2016 October; 32 (10): 1123-31).

An autophagy inhibiting peptide modulator of mTOR according to the invention is in particular provided for use in angiogenic control when said vasculopathy comprise a diabetic vasculopathy, in particular wherein said subject is having or suspected of having a C-peptide deficiency. Often such patients may already be treated with insulin (be it on-pump with a rapid-acting insulin, or subcutaneously with a rapid-, short-intermediate- or long-acting insulin, with other growth factors such as IGF, PDGF or VEGF, or growth factors related thereto, to combat any of the risks on blindness, end-stage renal failure or lower-extremity-amputations commonly associated with and thought resulting from diabetic vasculopathies. Such a modulator of mTOR find a particular advantageous use in patients that suffer from C-peptide deficiency, said deficiency for example present when said patient has a, preferably recurring, fasting C-peptide level of at least below 1 nmol/L, preferably at least below 0.5 nmol/L, more preferably at least below 0.3 nmol/L, more preferably at least below 0.2 nmol/L, more preferably at least below 0.1 nmol/L, more preferably at least below 0.06 nmol/L. In particular, the invention provides the use of autophagy inhibiting peptide-modulator of mTOR in the treatment of a vasculopathy, preferably a diabetic vasculopathy. In another embodiment, the invention provides the use of autophagy inhibiting peptide-modulator of mTOR in the treatment of a nephropathy, preferably a diabetic nephropathy. In yet another embodiment, the invention provides the use of autophagy inhibiting peptide-modulator of mTOR in the treatment of a retinopathy, preferably a diabetic retinopathy. In yet another embodiment, the invention provides the use of autophagy inhibiting peptide-modulator of mTOR in the treatment of a neuropathy, preferably a diabetic neuropathy.

On the other hand, the invention provides a growth factor formulation (formulations herein preferably comprising pharmaceutical formulations or pharmaceutical compositions), such as an insulin formulation, an IGF formulation, a PDGF formulation, a VEGF formulation, or a formulation of another growth factor useful in treatment of vascular disease, in particular useful in glycaemic control, having been provided with an autophagy inhibiting peptide-modulator of mTOR for the purposes of angiogenic control, as provided herein. A formulation of a growth factor and an autophagy inhibiting modulator of mTOR is provided, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), isoleucine (I), proline (P), arginine (R), and asparagine (N). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L), and proline (P). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), valine (V), leucine (L). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and valine (V). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), glycine (G), and leucine (L). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), glutamine (Q), leucine (L), and proline (P). In a preferred embodiment, the invention provides an insulin formulation with an autophagy inhibiting modulator of mTOR, said modulator comprising a source of amino acids, preferably a peptide, said amino acids for least 50%, preferably at least 75%, more preferably at least 80%, more preferably at least 85%, more preferably at least 90%, more preferably at least 95% selected from the group of alanine (in one letter code: A), valine (V), leucine (L), and proline (P). A formulation of an insulin comprising (with) an autophagy inhibiting modulator of mTOR according to the invention is preferred for use in inducing angiogenic activity in prevention or treatment of a vasculopathy of a subject, said subject also in need of achieving or maintain glycaemic control, in particularly preferred use in prevention or treatment of a nephropathy, for use in prevention or treatment of end-stage renal disease, for use in prevention or treatment of a retinopathy, for use in prevention or treatment of blindness or substantial loss (>30%-<90%) of sight, for use in prevention or treatment of a neuropathy, for use in prevention or treatment of a peripheral diabetic neuropathy, for use in prevention or treatment of an diabetic ulcer, in particular wherein said vasculopathy comprise a diabetic vasculopathy, especially when said subject is having or suspected of having a C-peptide deficiency, as defined herein. Such autophagy inhibiting peptide-modulators of mTOR and use thereof in growth factor formulations, as provided herein, typically comprise peptides and/or sources of amino acids that target the nutrient—in particular the amino acid—sensing system of the mechanistic target of rapamycine, mTOR, inhibit autophagy, and therewith induce angiogenic activities in a patient in need thereof. Typically, in the case of glycaemic control through the use of insulin formulations, insulin requirements (units/kg/day) are determined on the basis of patient's age, weight, and residual pancreatic insulin activity. Patients will typically require a total daily insulin dose of 0.4-1.0 units/kg/day and typical starting dose in metabolically-stable patients is 0.5 units/kg/day. However, as said, glycaemic control, even with insulin, is no substitute for C-peptide deficiency. It is herein provided to use an insulin formulation that also comprises an autophagy inhibiting peptide-modulator of mTOR in particular in patients that suffer from C-peptide deficiency, said deficiency for example present when said patient has a, preferably recurring, fasting C-peptide level of at least below 1 nmol/L, preferably at least below 0.5 nmol/L, more preferably at least below 0.3 nmol/L, more preferably at least below 0.2 nmol/L, more preferably at least below 0.1 nmol/L, more preferably at least below 0.06 nmol/L.

The invention therewith provides pharmaceutical formulations, methods and means to prevent or treat the occurrence of diabetic vasculopathy in patients, such as treatment of patients having nephropathy (predisposing for end-stage renal disease), retinopathy (predisposing for blindness) and/or neuropathy, in particular peripheral diabetic neuropathy and ultimately provides treatment of ulcer patients with vascular impairment, diabetic ulcers in particular predisposing for amputation of lower extremities in man. Typically, herein peptides are defined as having 50 or less amino acids, for the purpose of this disclosure, proteins are defined as having >50 amino acids. A autophagy inhibiting peptide-modulator of mTOR herein is defined as a linear, branched or circular string of no longer than 50 amino acids that comprises a peptide sequence with at least 50%, more preferably at least 75%, most preferably 100% amino acids selected from the group of autophagy inhibiting amino acids alanine (in one letter code: A), glutamine (Q), glycine (G), valine (V), leucine (L), proline (P), isoleucine (I), arginine (R) and asparagine (N). Molecular mode-of-action (MoA) of this group of peptides does not depend on their exact sequence. Instead, their constituent amino acids provide autophagy inhibiting signals to the nutrient-sensing system of mTOR; leading to inhibition of autophagy and resulting in proteogenesis with resolve of disease.

As reviewed in Sciarretta et al (New Insights Into the Role of mTOR Signaling in the Cardiovascular System. Circ Res. 2018 Feb. 2; 122 (3): 489-505) the mechanistic (previously called mammalian) target of rapamycin (mTOR) is an atypical serine/threonine kinase, belonging to the phosphoinositide kinase-related kinase (PIKK) family. It is an evolutionarily conserved protein that plays a central role in the regulation of cellular physiology, metabolism and stress responses Some call it the cellular master switch. mTOR interacts with specific adaptor proteins and forms two distinct macromolecular complexes, named mTOR complex 1 (mTORC1) and mTOR complex 2 (mTORC2). The previous paradigm in mTOR biology included involvement of mTOR in the regulation of protein synthesis, cellular growth and ribosomal biogenesis, and sensing and integrating different upstream inputs, such as growth factors, nutrients, amino acids, starvation and hypoxia. However, it is now known that the mTOR pathway also controls other important cellular processes, including cell survival, mitochondrial biogenesis and function, lipid synthesis and autophagy. The signalling network of mTORC2 is less characterized than that of mTORC1. Some studies have suggested that the activities of mTORC1 and mTORC2 are strongly interconnected, with mTORC2 being less sensitive to acute rapamycin treatment than mTORC1. Previous work also indicated that mTORC2 is involved in the regulation of cell survival, growth and proliferation, and controls cell architecture and polarity. Typically, amino acids are not only the building blocks of protein, but are also signalling molecules, as well as regulators of gene expression, metabolic processes and developmental changes in the body, with crucial roles of amino acids and their metabolites in the health and disease. Substantial evidence indicate that amino acids play a fundamental role in the vascular system. While amino acids serve as basic building blocks for protein synthesis and constitute an important energy source, a select group has been widely studied in the context of vascular disease. mTOR, in particular mTORC1, is a critical kinase that regulates cell growth and proliferation, by sensing nutrients such as amino acids and glucose. Alanine and glutamine are the most abundant amino acids circulating in the blood. Amino acids not only participate in intermediary metabolism but also stimulate insulin-mechanistic target of rapamycin (MTOR)-mediated signal transduction which controls the major metabolic pathways. Among these is the pathway of autophagy which takes care of the degradation of long-lived proteins and of the elimination of damaged or functionally redundant organelles. Proper functioning of this process is essential for cell survival. Dysregulation of autophagy has been implicated in the aetiology of several pathologies. Clearly, only certain amino acids are able to modulate autophagy, and their functions are highly cell-specific. However, many of the details of the relationship between certain amino acids and cellular metabolism are unclear (see further FIG. 5).

### Further Embodiments

1 A method for identifying a source of, preferably L-proteinogenic, amino acids, preferably a peptide, capable of inducing angiogenic activity, comprising providing cells with a peptide comprising L-proteinogenic amino acids, said amino acids for least 50% selected from the group of alanine (in one letter code: A), glutamine (Q), glycine (G), valine (V), leucine (L), isoleucine (I), proline (P) and arginine (R), and determining angiogenic activity in a sprouting assay.
2 A method for inducing angiogenic activity, comprising providing cells with a source of, preferably L-proteinogenic, amino acids, said amino acids for at least 50% selected from the group of alanine (in one letter code: A), glutamine (Q), glycine (G), valine (V), leucine (L), isoleucine (I), proline (P) and arginine (R).
3 A method for inducing angiogenic activity, comprising providing cells with a peptide as a source of, preferably L-proteinogenic, amino acids, said peptide consisting of amino acids that are for at least 50% selected from the group of alanine (in one letter code: A), glutamine (Q), glycine (G), valine (V), leucine (L), isoleucine (I), proline (P) and arginine (R).
4 A method according to further embodiment 2 or 3, wherein said source of, preferably L-proteinogenic, amino acids, preferably a peptide, is identifiable with a method according to further embodiment 1.
5 A method according to anyone of further embodiments 1 to 4 wherein said amino acids are for at least 50% selected from the group of alanine (in one letter code: A), glutamine (Q), glycine (G), valine (V), leucine (L), and proline (P).
6 A method according to anyone of further embodiments 2 to 5 wherein said source or peptide has an amino acid sequence that is derived from a peptide or protein shown or suspected of having angiogenic activities.
7 A method according to further embodiment 6 wherein said peptide has an amino acid sequence that is derived from a chorionic gonadotropin.
8 A method according to further embodiment 6 wherein said peptide has an amino acid sequence that is derived from a C-peptide.
9 A method according to further embodiment 6 wherein said peptide has an amino acid sequence that is derived from a galectin-3.
10 A method according to further embodiment 6 wherein said peptide has an amino acid sequence that is derived from a luteotropin.
11 A method according to further embodiment 6 wherein said peptide has an amino acid sequence that is derived from an extra-cellular matrix protein.
12 A method according to further embodiment 12 wherein said extra-cellular matrix protein is an elastin or collagen.
13 A method according any one of further embodiments 1 to 12 wherein said peptide comprises less than 31 amino acids, preferably less than 26 amino acids, more preferably less than 21 amino acids, more preferably less than 16 amino acids, more preferably less than 13 amino acids.
14 A method according any one of further embodiments 1 to 13 wherein said peptide comprises more than 3 amino acids, preferably more than 5 amino acids, more preferably more than 7 amino acids, more preferably at least 9 amino acids.
15 A method according to any one of further embodiments 1 to 14 wherein said cells are also provided with a compound capable of glycaemic control such as an insulin or derivative thereof.
16 A method according to further embodiment 15 wherein said insulin is a rapid-acting insulin.
17 A method according to further embodiment 16 wherein said source comprises a peptide that comprises more than 3 amino acids and less than 13 amino acids.
18 A method according to further embodiment 15 wherein said insulin is a short-acting insulin.
19 A method according to further embodiment 18 wherein said source comprises a peptide that comprises more than 4 amino acids and less than 21 amino acids.
20 A method according to further embodiment 15 wherein said insulin is an intermediate-acting insulin.
21 A method according to further embodiment 20 wherein said source comprises a peptide that comprises more than 6 amino acids and less than 26 amino acids.
22 A method according to further embodiment 15 wherein said insulin is a long-acting insulin.
23 A method according to further embodiment 22 wherein said source comprises a peptide that comprises more than 7 amino acids and less than 31 amino acids.
24 A method according to any one of further embodiments 1 to 23 wherein said peptide is provided as a salt of an organic acid, preferably wherein said peptide is provided as a salt of maleic acid, more preferably of acetic acid, more preferably of tartaric acid and most preferably of citric acid.
25 A method according to any one of further embodiments 1 to 24 wherein said cells are of human origin.
26 A method according to any one of further embodiments 1 to 25 wherein said peptide is of human origin.
27 Use of a source of, preferably L-proteinogenic, amino acids, preferably a peptide, said amino acids for at least 50% selected from the group of alanine (in one letter code: A), glutamine (Q), glycine (G), valine (V), leucine (L), isoleucine (I), proline (P) and arginine (R) for the production of a pharmaceutical composition for treatment of a subject having or suspected of having a vasculopathy, preferably a diabetic peripheral neuropathy.
28 Use according to further embodiment 26 wherein said subject has or is at risk of developing a diabetic ulcer, preferably a foot ulcer.
29 Use according to further embodiment 26 or 27 wherein said subject has or is suspected to have a C-peptide deficiency.
30 Use according to any one of further embodiments 26 to 28 wherein said subject has a fasting C-peptide level of at least below 1 nmol/L, preferably at least below 0.5 nmol/L, more preferably at least below 0.3 nmol/L, more preferably at least below 0.2 nmol/L, more preferably at least below 0.1 nmol/L, more preferably at least below 0.06 nmol/L.
31 Use according to any one of further embodiments 26 to 29 wherein said peptide is provided as a salt of an organic acid, preferably wherein said peptide is provided as a salt of maleic acid, more preferably of acetic acid, more preferably of tartaric acid and most preferably of citric acid.
32 Use according to any one of further embodiments 26 to 30 wherein said treatment comprises a method for inducing angiogenic activity according to any one of further embodiments 2 to 25.
33 A source of, preferably L-proteinogenic, amino acids, preferably a peptide, said amino acids for at least 50% selected from the group of alanine (in one letter code: A), glutamine (Q), glycine (G), valine (V), leucine (L), isoleucine (I), proline (P) and arginine (R) in treatment of a subject having or suspected of having a vasculopathy, preferably a diabetic peripheral neuropathy.
34 A source according to further embodiment 30 wherein said subject has or is at risk of developing a diabetic ulcer, preferably a foot ulcer.
35 A source according to further embodiment 30 or 31 wherein said subject has or is suspected to have a C-peptide deficiency.
36 A source according to any one of further embodiments 32 to 34 wherein said subject has a fasting C-peptide level of at least below 1 nmol/L, preferably at least below 0.5 nmol/L, more preferably at least below 0.3 nmol/L, more preferably at least below 0.2 nmol/L, more preferably at least below 0.1 nmol/L, more preferably at least below 0.06 nmol/L.
37 A source according to any one of further embodiments 32 to 35 wherein said peptide is provided as a salt of an organic acid, preferably wherein said peptide is provided as a salt of maleic acid, more preferably of acetic acid, more preferably of tartaric acid and most preferably of citric acid.
38 A source according to any one of further embodiment 30 to 33 wherein said treatment comprises a method for inducing angiogenic activity according to any one of further embodiments 2 to 24.
39 A method of treatment of a subject having or suspected of having a vasculopathy, preferably a diabetic peripheral neuropathy, more preferably wherein said subject has or is at risk of developing a diabetic ulcer, preferably a foot ulcer, said method comprising providing said subject with a source of amino acids according to any one of further embodiments 30 to 34.

### LEGENDS TO THE DRAWINGS

FIG. 1

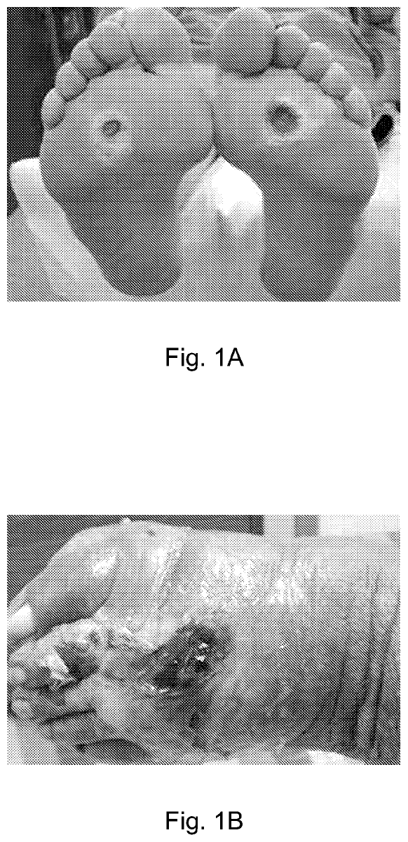

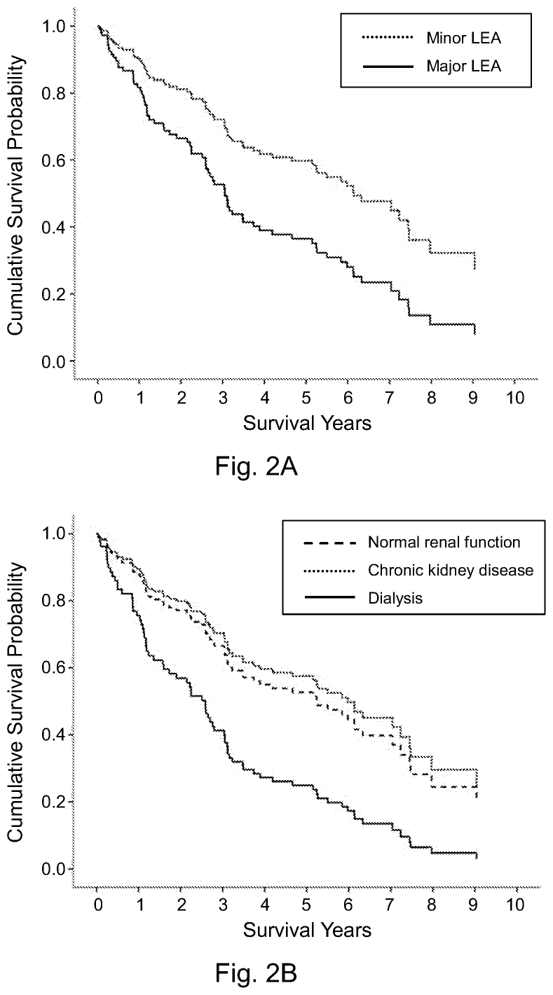

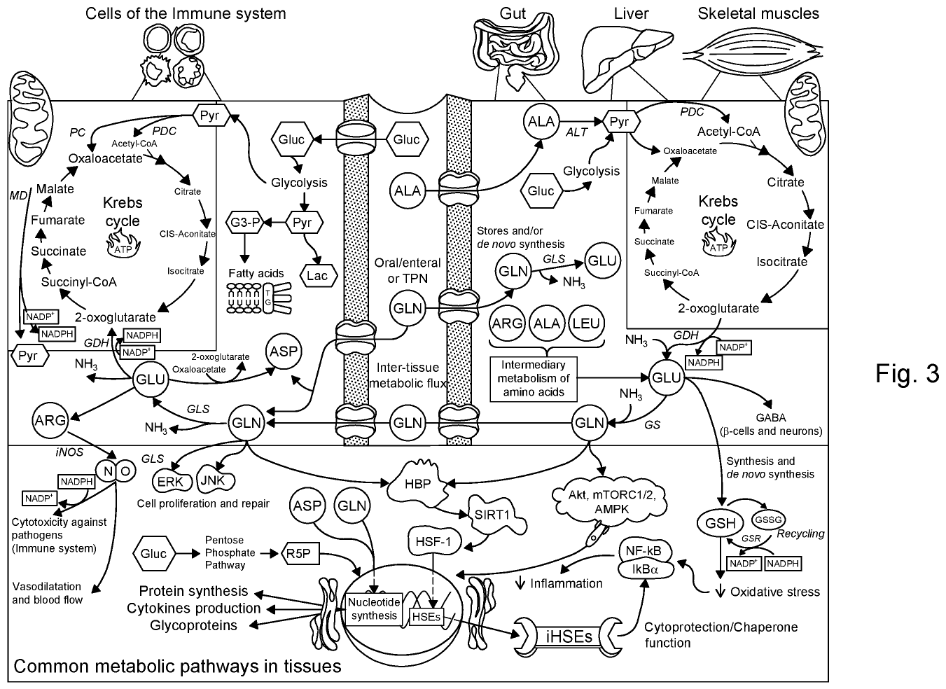

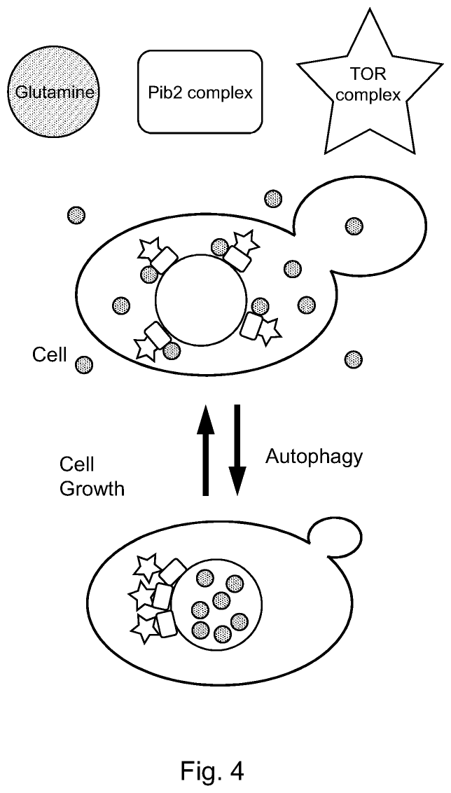

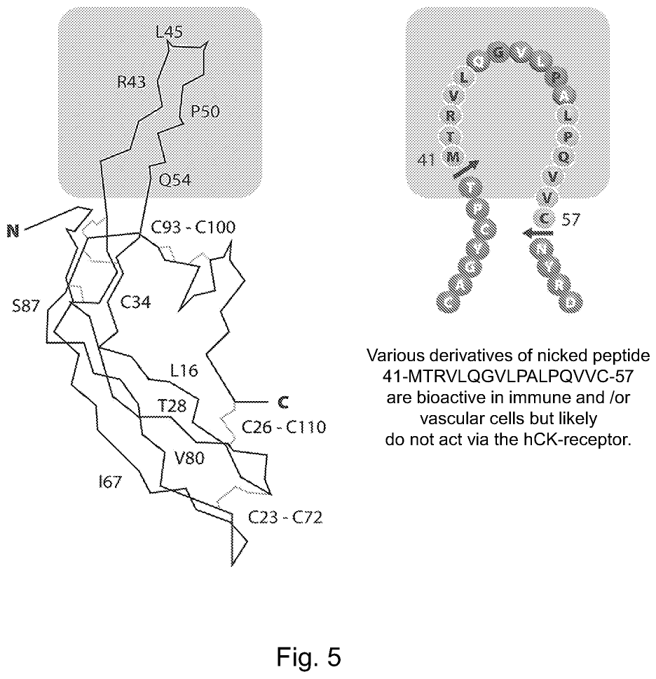

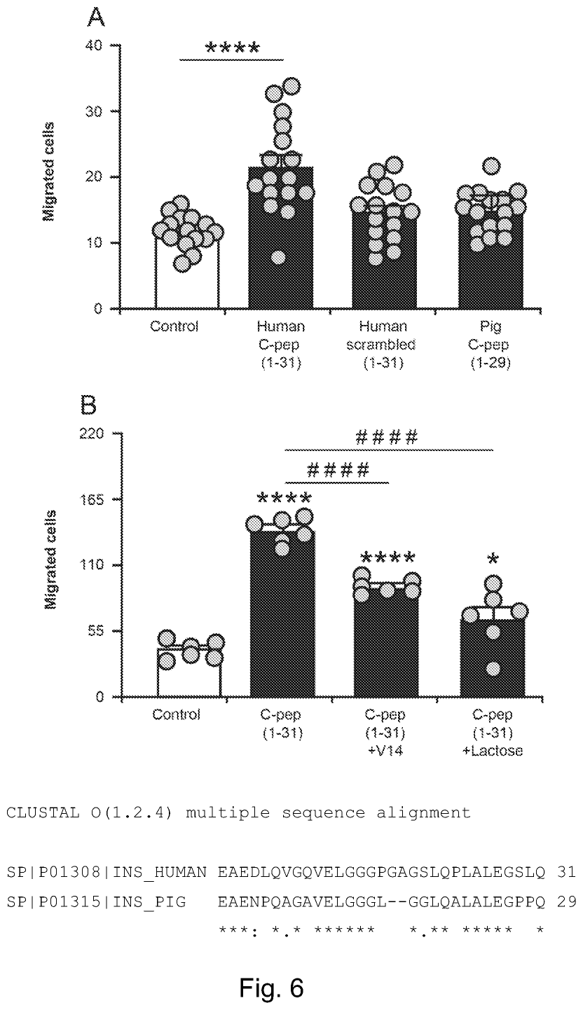

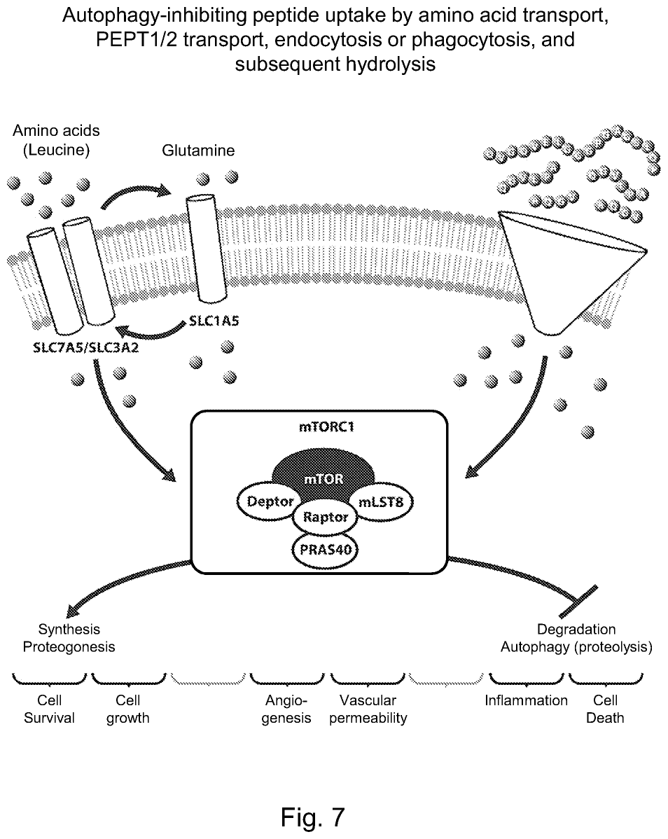

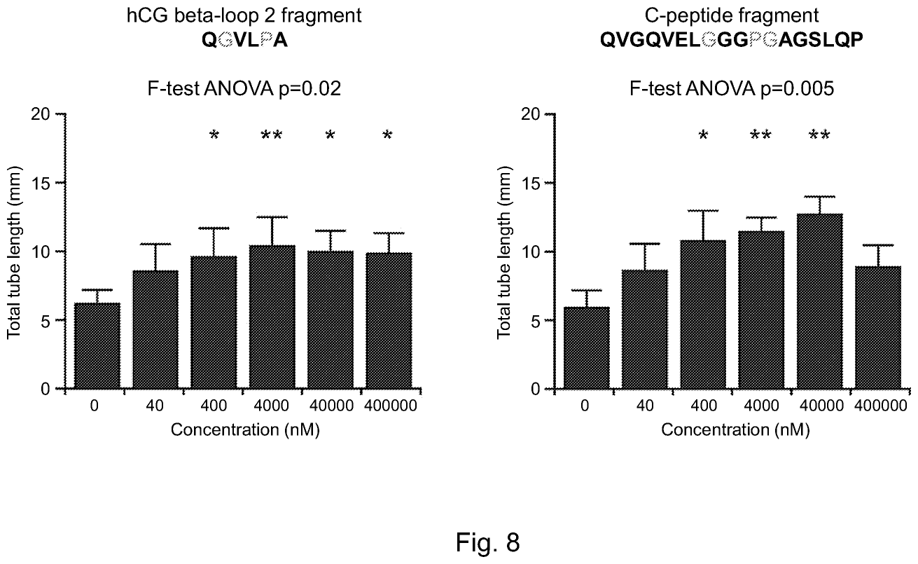

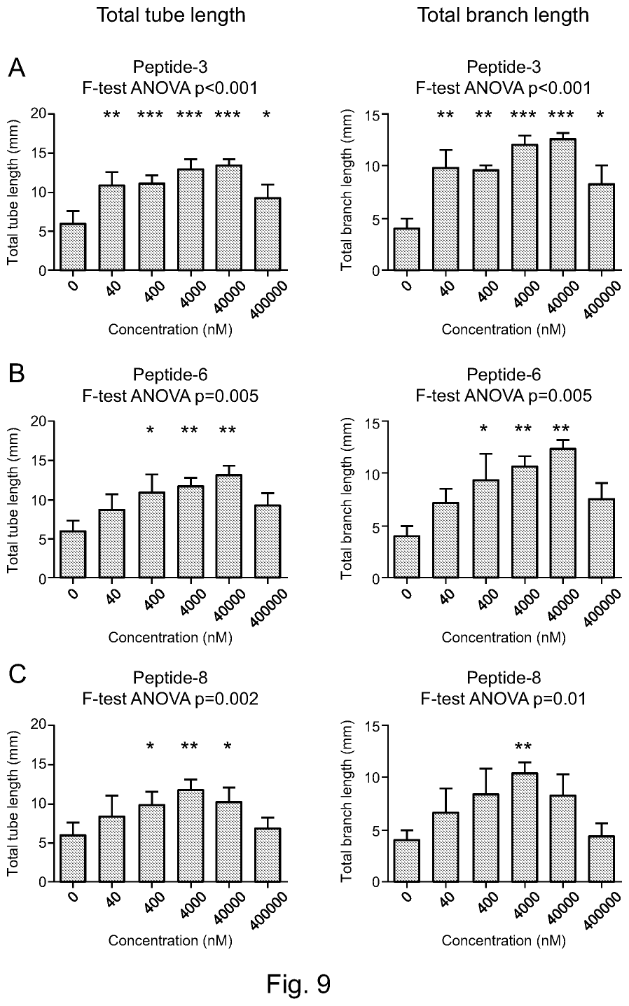

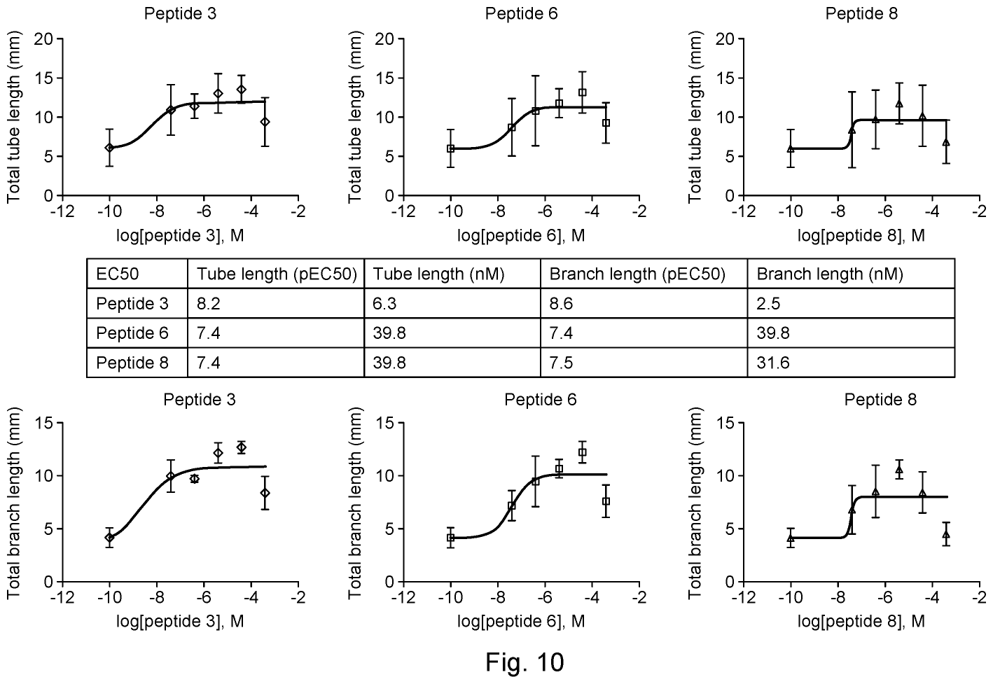

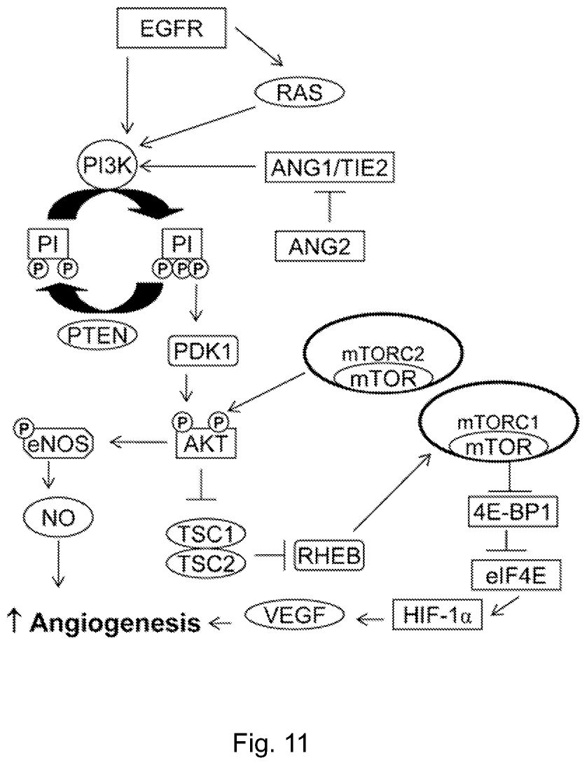

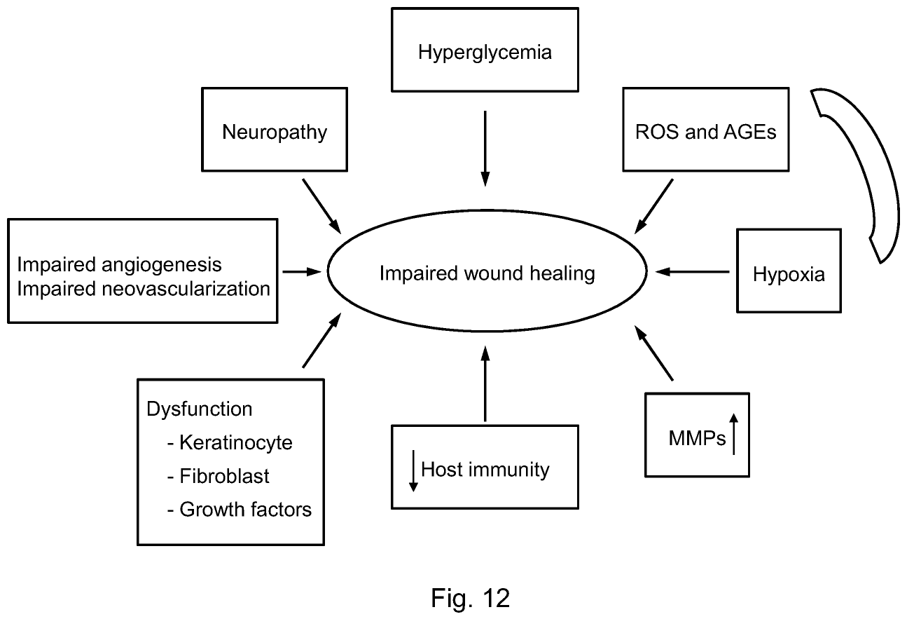

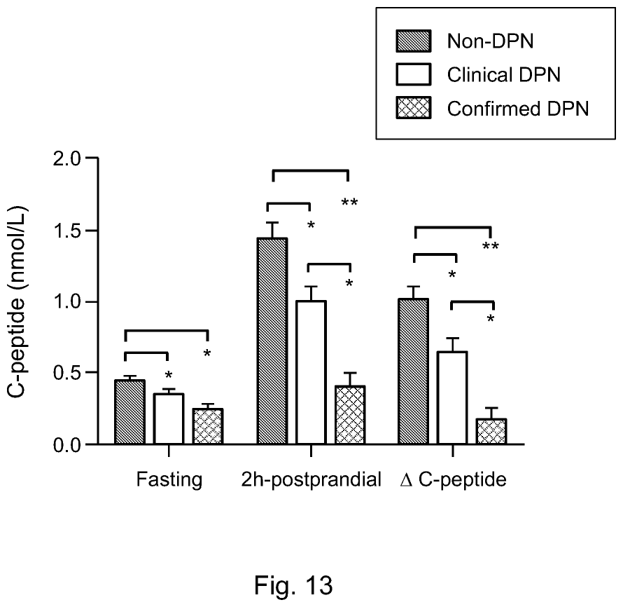

The need for angiogenic control, concomitant with glycaemic control, is particularly well illustrated in the case of diabetic vasculopathies with diabetic peripheral neuropathy (DPN) that invariably develop in a large set of patients suffering from type 1 diabetes or type 2 diabetes with C-peptide deficiency. DPN predisposes for foot ulcers that that may develop in t foot gangrene. It ranges between (FIG. 1A) ulcers with developing dry gangrene, ischemic and necrotic tissue but no infection, and (FIG. 1B) wet gangrene with both infectious and necrotic tissue. Dry gangrene can lead up to wet gangrene. In the more pronounced stages of dry gangrene than portrayed here, the affected area is often characterized by a dark green or purple, almost black colour. The skin may be dry and wrinkled due to the lack of oxygen. Wet gangrene has a wet appearance. This type is characterized by blisters and swelling, indicating fulminant inflammation.

FIG. 2

Similarly, the need for angiogenic control, concomitant with glycaemic control, is particularly well illustrated in the case of diabetic vasculopathies with diabetic peripheral neuropathy (DPN) and nephropathies that ultimately develop in a large set of patients suffering from type 1 diabetes or type 2 diabetes with C-peptide deficiency. Adjusted survival curve from a Cox proportional hazard model according to lower-extremity amputation (LEA) status and renal function status. 2A Compared to the minor LEA group, the adjusted hazard ratio for mortality was 1.80 (95% CI 1.05-3.09, P=0.03) for those with major LEAs. 2b Compared to the normal renal function group, the adjusted hazard ratio for mortality was 0.87 (95% CI 0.48-1.59, P=0.66) for those with CKD and 2.09 (95% CI 0.99-4.41, P=0.05) for those undergoing dialysis.

FIG. 3

Glutamine inter-tissue metabolic flux starting in skeletal muscle, liver, and gut continues in the immune cells. Abbreviations: Glutamine, GLN; glutamate, GLU; aspartate, ASP; arginine, ARG; leucine, LEU; alanine, ALA; glucose, Gluc; pyruvate, Pyr; pyruvate dehydrogenase; PDC; pyruvate carboxylase, PC; malate dehydrogenase, MD; glyceraldehyde-3-Phosphate, G3-P; lactate, Lac; triacylglycerol, TG; ribose 5-phosphate, R5P; alanine aminotransferase, ALT; glutamate dehydrogenase, GDH; glutamine synthetase, GS; glutaminase, GLS; inducible nitric oxide synthase, iNOS; intracellular heat shock protein, iHSP; heat Shock Factor 1, HSF-1; heat shock elements, HSEs; sirtuin 1, SIRT1; hexosamine biosynthetic pathway, HBP; ammonia, NH3; glutathione, GSH; oxidized GSH, GSSG; glutathione S-reductase, GSR; protein kinase B, Akt; AMP-activated protein kinase, AMPK; mTOR complex 1 and 2, mTORC1/2, extracellular signal-regulated kinases, ERK; c-Jun N-terminal kinases, JNK; gamma-Aminobutyric acid, GABA.

FIG. 4

mTOR complex activation mechanism by glutamine binding to Pib2 complex bound directly to glutamine activates TOR complexes, induces cell growth and inhibits autophagy. When glutamine is available, cells start to grow, while unavailable, they start autophagy.

FIG. 5

The elastin-receptor-complex (ERC) binding motif xGxxPx is present in a peptide fragment derived from position ˜41-57 in loop 2 of β-human chorionic gonadotrophin (beta-hCG).

Left: Structure of β-human chorionic gonadotrophin (beta-hCG) with loop 2 indicated in grey, as adapted from Lapthorn A J, Harris D C, Littlejohn A, Lustbader J W, Canfield R E, Machin K J, et al. Crystal structure of human chorionic gonadotropin. Nature. 1994; 369 (6480): 455-61.

Top right: Amino acid sequence of loop 2 of beta-hCG. Its xGxxPx motif is represented by hexapeptide QGVLPA. Various ‘nicked’ or fragmented peptides of beta-hCG derive by proteolysis from the peptide backbone between amino acids 40 to 54 (Birken S, Berger P, Bidart J-M, Weber M, Bristow A, Norman R, et al. Preparation and Characterization of New WHO Reference Reagents for Human Chorionic Gonadotropin and Metabolites. Clinical Chemistry. 2003; 49 (1): 144-54). All amino acid sequences are represented in the one-letter-code.

Bottom right: Various synthetic peptides derived from a nicked fragment of loop 2, such as MTRVLQGVLPALPQ, circular CVLQGVLPALPQVVC, VLQGVLPALPQVVC, VLQGVLPALPQ, LQGV, AQGV, VLPALP and VLPALPQ are bioactive when tested in immune and/or vascular cells. A receptor for those fragments has hitherto been undisclosed.

FIG. 6

Activity of human C-peptide depends on the xGxxPG motif and is blocked by antagonists of ERC.

A: Synthetic human-C-peptide (1-31, having ERC-binding-motif LGGGPG) was tested in human CD4+ lymphocyte-migration assay together with negative and treatment controls: no C-peptide added, scrambled version of human C-peptide (1-31, lacking any xGxxPx motif) and pig-C-peptide (1-29, ˜74% similarity to human C-peptide but lacking any xGxxPx motif; alignment with human C-peptide shown herewith).

Freshly isolated human CD4+ cells were incubated for 3 h with the indicated C-peptide variants (10 nM) to assess the effect on cell migration in a modified Boyden chamber. Results are expressed as the mean±SEM, ****P<0.001 treatments vs. control.

B: Freshly isolated human CD4 cells were preincubated for 3 h with V14 peptide (10 μM) or lactose (10 mM) and then stimulated with C-peptide (1-31) for 3 H as indicated in (A). Results are expressed as the mean±SEM. *P<0.05, ****P<0.001 treatments vs. control, 190 ###P<0.001 treatments vs. human C-peptide (1-31).

A CLUSTAL O (1.2.4) multiple sequence alignment of human and pig C-peptide is shown to illustrate lack of ERC-binding motif in pig C-peptide versus its presence in human C-peptide.

FIG. 7

Nutrient sensing is important to sustain normal cell growth and proliferation. mTOR complex 1 (mTORC1) senses nutrients to regulate cell growth, autophagy, and other mTORC1-mediated processes. Growth factors such as insulin, IGF-1, PDGF and VEGF, amino acids, energy status, and stress control mTORC1. Amino acids are essential for mTORC1 activation. Growth factors alone cannot attain maximal mTORC1 activity without amino acid supplementation (Sancak et al., The Rag GTPases bind raptor and mediate amino acid signaling to mTORC1. Science. 2008 Jun. 13; 320 (5882): 1496-501). Increased amino acid concentrations promote mTORC1 lysosomal localization and subsequent activation. Typically, some amino acids activate mTOR more than others, and therewith inhibit autophagy more than others. Autophagy-inhibiting peptide modulators of mTOR preferentially comprise peptides having a relative increased content of those autophagy-inhibiting amino acids, to activate mTOR best. Such peptides (shown here QVGQVELGGGPGAGSLQP (derived from human C-peptide), QGVLPA or AQGV (both derived from hCG) as well as others disclosed in this application) may enter the cell by common mechanisms of extracellular hydrolyzation and uptake by various amino acid transporters, as depicted at top-left. Intracellular glutamine also has the capacity to exchange with extracellular essential amino acids https://www.nature.com/articles/ncomms11457-ref-CR14, in particular leucine. Depicted at top right is peptide entry. Extracellular di- and tri-peptides may enter via peptide transporters (PEPT1/2) regulated transport. Larger peptides may enter via (receptor mediated) endocytosis or phagocytosis. Arginine-rich cell-penetrating peptides may passively enter vesicles and live cells by inducing membrane multilamellarity and fusion. Further intracellular hydrolyzation of autophagy-inhibiting peptide liberates autophagy inhibiting amino acids that modulate mTOR and signals a multitude of processes that balance between metabolic synthesis (proteogenesis) or degradation (autophagy) ranging from cell survival to cell death.

FIG. 8

Microvascular angiogenesis (sprouting) assay of human pulmonary microvascular endothelial cells grown in Matrigel and in response to increasing concentrations of peptides hexapeptide QGVLPA (derived from hCG) and 18-meric peptide QVGQVELGGGPGAGSLQP (derived from human C-peptide). Data shown are total tube length measurements at t=24 h. *p<0.05; **p<0.01. Hexapeptides VGVAPG (derived from human elastin), LGGGPG (derived from human C-peptide), and PGAYPG (derived from human galectin-3) also significantly induced angiogenic activities (not shown) in this experiment.

FIG. 9

Microvascular angiogenesis (sprouting) assay of human pulmonary microvascular endothelial cells grown in Matrigel and in response to increasing concentrations of peptides 3 (18-meric peptide VGVAPGVGVAPGVGVAPG, derived from human elastin), 6 (18-meric peptide QVGQVELGGGPGAGSLQP, derived from human C-peptide) and 8 (18-meric peptide, GAYPGAPGAYPGAPAPGV derived from the human N-terminal fragment of galectin-3), (FIGS. 9a, 9b and 9c, respectively). Data shown are total tube length measurements and total branch length measurements at t=24 h. *p<0.05; **p<0.01; ***p<0.001.

FIG. 10

Determination of EC50, the pharmacological concentration required to obtain a 50% angiogenic activity in tube- or branch-formation of the autophagy-inhibiting peptide modulators of mTOR VGVAPGVGVAPGVGVAPG derived from human elastin, QVGQVELGGGPGAGSLQP derived from human C-peptide and GAYPGAPGAYPGAPAPGV derived from human galectin-3, as tested in FIG. 9.

FIG. 11

Skin wound healing is a comprehensive and complex process involving inflammation response, angiogenesis, new tissue formation, and tissue remodelling that consists of proliferation and migration of various cell types (inflammatory cells, keratinocytes, endothelial cells (EC), fibroblasts, platelets) to finally restore the integrity of the skin barrier. Angiogenesis in wound-healing involves modulation of angiopoetin 1 (ANG1) versus angiopoetin 2 (ANG2), therewith also including the PI3K/AKT/mTOR pathway in angiogenesis. The PI3K/AKT/mTOR pathway modulates the expression of angiogenic factors such as nitric oxide and angiopoietins. Numerous inhibitors of the PI3K/AKT/mTOR pathway have been developed, and these agents have been shown to decrease VEGF secretion and angiogenesis and there with hamper wound healing. To the contrary, activation of mTORC1 has indeed been shown to promote angiogenesis (Karar J, Maity A. PI3K/AKT/mTOR Pathway in Angiogenesis. Front Mol Neurosci. 2011 Dec. 2; 4:51). Autophagy-inhibiting peptide modulators of mTOR as provided herein activate mTOR and there with increase VEGF secretion and angiogenesis in wound healing.

FIG. 12

Further factors contributing to decreased wound healing include hyperglycaemia, illustrating the importance of maintaining proper glycaemic control.

FIG. 13

Determination of C-Peptide Deficiencies.

Qiao, et al. (C-peptide is independent associated with diabetic peripheral neuropathy: a community-based study. Diabetol Metab Syndr 9, 12 (2017).) studied the relationship between residual C-peptide and DPN in patients with type 2 diabetes. With increased diabetes duration, the islet function diminishes gradually, resulting in reduced C-peptide and insulin levels and the prevalence of DPN increases. Fasting C-peptide, 2-h postprandial C-peptide and ΔC-peptide (i.e., 2-h postprandial C-peptide minus the fasting C-peptide) serum concentrations in the non-DPN group were significantly higher than those in the clinical DPN group (all P≤0.040) and the confirmed DPN group (all P<0.002).

### AUTOPHAGY INHIBITING PEPTIDES

### One Letter Code

In describing protein or peptide composition, structure and function herein, reference is made to amino acids. In the present specification, amino acid residues are identified by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

### Peptides

Peptide shall mean herein a natural biological or artificially manufactured (synthetic) short chain of amino acid monomers linked by peptide (amide) bonds. Glutamine peptide shall mean herein a natural biological or artificially manufactured (synthetic) short chain of amino acid monomers linked by peptide (amide) bonds wherein one of said amino acid monomers is a glutamine. Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided.

### Peptide Synthesis

In this application, peptides are either synthesized by classically known chemical synthesis on a solid support (Ansynth BV, Roosendaal, The Netherlands) or in solution (Syncom BV, Groningen, The Netherlands and Diosynth BV, Oss, The Netherlands). Pharmaceutical peptide compositions may be synthesized using trifluoroacetate as a counter-ion or salt after which trifluoroacetate is exchanged by a counter-ion such as maleate (from maleic acid), acetate (from acetic acid), tartrate (from tartaric acid) or citrate (from citric acid). The drug substance of AQGV (EA-230) for use in pre-clinical and clinical human studies has been manufactured by Organon N.V (formerly Diosynth B.V.), (Oss, The Netherlands), whereas filling and finishing of the final product has been performed by Octoplus Development, Leiden (The Netherlands). Molecular weight of EA-230 (AQGV) is 373 g/mol).

### Determination of Chemotactic Activity.

Human U937 monocytic cells are purchased from the American Type Culture Collection (ATCC catalog number CRL-1593.2, Manassas, Va). Cells are maintained in suspension culture in T-75 flasks containing RPMI 1640 medium supplemented with 10% fetal calf serum and antibiotics, and cultures are split every 3 to 5 days. Three days before use in chemotaxis assays, U937 cells are stimulated to differentiate along the macrophage lineage by exposure to 1 mmol/L dibutyryl cyclic adenosine monophosphate (dbcAMP; Sigma Chemical Co), as described. Cells are washed three times to remove culture medium and then resuspended in chemotaxis medium (Dulbecco's modified essential medium supplemented with 1% lactalbumin hydrolysate) for plating into assay chambers at a final concentration of 2.5×106 cells/mL. Chemotaxis assays are performed in 48-well microchemotaxis chambers (Neuro Probe, Cabin John, Md). The bottom wells of the chamber are filled with 25 ml of the chemotactic stimulus (or medium alone) in triplicate. An uncoated 10-mm-thick polyvinylpyrrolidone-free polycarbonate filter with a pore size of 5 mm is placed over the samples (Neuro Probe). The silicon gasket and the upper pieces of the chamber are applied, and 50 ml of the monocyte cell suspension are placed into the upper wells. Chambers are incubated in a humidified 5% CO2 atmosphere for 3 hours at 37° C., and nonmigrated cells are gently wiped away from the upper surface of the filter. The filter is immersed for 30 seconds in a methanol-based fixative and stained with a modified Wright-Giemsa technique (Protocol Hema 3 stain set; Biochemical Sciences, Inc, Swedesboro, NJ) and then mounted on a glass slide. Cells that are completely migrated through the filter are counted under light microscopy, with 3 random high-power fields (HPF; original magnification ×400) counted per well. Human monocytes are isolated from freshly drawn blood of healthy volunteers using serial Ficoll/Pelastin receptor complex (ERC)oll gradient centrifugation, as described elsewhere. Cells are cultured for 16 hours in RPMI-1640 media supplemented with 0.5% human serum to become quiescent after isolation. Purity of the cells is >95% as determined by flow cytometry analysis. Monocyte chemotaxis is assayed in a 48-well microchemotaxis chamber (Neuroprobe, Gaithersburg, MD) in serum-free media. Wells in the upper and lower chamber are separated by a polyvinylpyrrolidone-free polycarbonate membrane (pore size 5 μm; Costar). Freshly isolated monocytes at a density of 5×105/ml are incubated for 2.5 hours with recombinant C-peptide (Sigma), before migrated cells on the bottom face of the filter are stained and counted under the light microscope. Maximal chemotactic activity is measured with 0.1 mmol/L N-formyl-methionyl-leucyl-phenylalanine (f-MLF; Sigma Chemical Co), and checkerboard analysis is used to distinguish chemotaxis from chemokinesis.

### Cell Isolation

Blood is drawn from healthy volunteers into tubes containing citrate as an anticoagulant. Neutrophils are isolated by using a Polymorphprep kit (Nicomed, Oslo, Norway) according to the manufacturer's instructions; monocytes are purified with magnetic beads (Miltenyi Biotech). The purity of the cells, as assessed by flow cytometry (anti-CD45, 14, DR, and CD66b), is >93%. For each cell type, samples from two different donors are examined.

Distribution and Elimination of Intravenously Injected [¹⁴C]-AQGV from Mice

The present study, which was conducted by TNO Biosciences, Utrechtseweg, Netherlands, was designed to provide data on the distribution and metabolism of [¹⁴C]-AQGV (Ala-Gln-[1-¹⁴C]Gly-Val) in male CD-1 mice following a single intravenous dose of 50 mg/kg. To this end, mice were sacrificed at 10, 30 and 60 minutes, and 6 and 24 hours after administration of radiolabeled AQGV, counts in various tissues were determined, and the radioactivity present in the urine and plasma were analyzed by HPLC.

There was relatively little radioactivity in the blood 10 minutes after the radiolabeled peptide was injected. If all of the counts were in intact peptide, the amount present would be 17.2 μg/g. No parent compound could be detected in plasma after 10 minutes however; [¹⁴C]-AQGV appeared to be hydrolyzed quite rapidly. No parent compound was detected in the urine either. The radioactivity in urine was mostly present as hydrophilic compounds, eluting in or just after the dead volume of the HPLC column.

The radioactivity present in various organs exceeded that in the blood. The highest concentrations of “peptide” after 10 minutes were found in the kidneys (362 μg/g), liver (105 μg/g), testis (85.7 μg/g), lung (75.2 μg/g), and spleen (74.7 μg/g). In general, a gradual decrease in radioactivity was observed thereafter. After 24 hours, the highest concentrations were found in kidneys (61.9 μg/g), thymus (43.1 μg/g), spleen (39.3 μg/g), liver (37.6 μg/g), and skin (37.5 μg/g).

The average total recoveries of radioactivity 10, 30, and 60 minutes, and 6 and 24 hours after dose administration were 83.2, 70.5, 62.9, 52.6 and 50.8%, respectively. These results strongly indicate that there is rapid formation of [¹⁴C]-volatiles after dose administration, most likely ¹⁴C—CO2, which is exhaled via the expired air. After 24 hours, 10.2% of the administered radioactivity was excreted in urine, and 2.6% in feces.

### Conclusions

After intravenous injections, [¹⁴C]-AQGV was rapidly removed from the blood. This is consistent with the results of pharmacokinetic studies that are presented below. Metabolite profiles in blood plasma and urine revealed no parent compound, indicating rapid metabolism of [¹⁴C]-AQGV. About 50% of the administered radioactivity was exhaled as volatiles, most likely ¹⁴C—CO2, up to 24 hours. The results of the present study indicate rapid hydrolysis of [¹⁴C]-AQGV yielding [1-¹⁴C]-glycine, which is subsequently metabolized into ¹⁴C—CO2 and exhaled in the expired air. The absence of parent compound in plasma and urine suggests that the radioactivity present in tissues and organs could be present only as hydrolyzation products of the metabolism of [¹⁴C]-AQGV.

### Peptide Hydrolysis

The disclosure provides that when a peptide provide with autophagy inhibiting amino acids such as peptide AQGV encounters a cell, the peptide is hydrolyzed, be it extracellular at the surface of that cell, or after endocytosis, in the case of vascular cells for example by elastin receptor mediated endocytosis, of the peptide by the cell in the phagolysosome. Many peptidases are known to exist on or in cells that can rapidly hydrolyze peptides, and continued hydrolysis invariably leads to tripeptides and dipeptides. Likewise, hydrolysis in the lysosomes by tripeptidyl and dipeptidyl peptidase will equally result in single amino acids. Studies with ¹⁴C labeled AQGV have factually shown full hydrolysis of the peptide in 15 min after its administration in mice. Tripeptides, dipeptides and single amino acids will result from the hydrolysis of AQGV, or its sister compound LQGV, or for that matter from any other suitable oligopeptide, when presented to a cell.

### Peptide Transport

Similarly, several studies have reported the role of p38 MAPK in survival of different type of mature granulocytes. Granulocytes (e.g. neutrophils, eosinophils, basophils) have in common their terminal differentiation stage. These cells have fragmented nuclei and have an accumulation of granules containing preformed secretion factors. It is herein been proposed that p38 MAPK is required for survival of neutrophils, and inactivation of p38 MAPK is essential for death and the elimination of these cells as well as that p38 MAPK is required for contraction of endothelial cells, and inactivation of p38 MAPK is essential for relaxing those vascular cells so that those can restore vascular wall integrity, as well as inactivation of p38 MAPK activity is essential for pacifying neutrophils, and other leucocytes cells exploring the vascular permeability of vascular endothelial blood vessel wall.

Di- and tripeptides are selectively transported via the PEPT1/2 transporters. Tripeptides, dipeptides and single amino acids are actively transported through the cell membrane, whereby uptake of dipeptides and tripeptides involves a separate mechanism than uptake of single amino acids, namely via the PEPT1 and PEPT2 transporters. Potentially all 400 di- and 8,000 tripeptides can be transported by PepT1 and PEPT2. Intestinal cell transport of amino acids in the form of peptides was demonstrated to be a faster route of uptake per unit of time than their constituent amino acids in the free form (reviewed in J Anim Sci, 2008; 9, 2135-2155).

mTOR is Involved

Finally, we propose involvement of mechanistic target of rapamycine, mTOR. In this perspective, the peptide enters cells either via PEPT1/2 or by active endocytosing or phagocytosing processing, after which the peptide is fully hydrolyzed in the phagolysosome and the resulting autophagy inhibiting amino acids are presented to mTOR complex where they cause inhibition of autophagy of the cell. Tetrapeptide, tripeptide and dipeptide activities may all reflect the final causal activity of single amino acids A, Q, G, V, selected from the group of amino acids A,Q,G,V,L and P. In this way, the amino acids A,Q,G,V,L and P are food for mTOR. Indeed, preliminary results show similar effects on inhibition of the p38 pathway when different tri- and dipeptides derived from AQGV in an FPR-signaling assay are used. Individual amino acids may approach mTOR via the cytosol, but amino acids in peptide fragments (strings such as AQGV), likely enter the mTOR machinery via the phagolysosome. Activation of mTOR by amino acids in sources such as peptides can therefore be explained from two perspectives, 1) whereby endocytosis of peptide strings is paramount for all phagocytosing cells, such as neutrophils and monocytes, 2) whereby peptide fragments enter via PEPT1/2.

Amino Acids Activate mTOR Pathways and Inhibit Autophagy

Autophagy serves to produce amino acids for the survival of a cell when nutrients fall short, and amino acids are effective inhibitors of autophagy. Mechanistic-target-of-rapamicin (mTOR) is a critical regulator of autophagy induction, with activated mTOR suppressing autophagy and negative regulation of mTOR promoting it. Amino acids are indeed considered important regulators of mTOR complex 1 or 2 activation, affecting cell proliferation, protein synthesis, autophagy and survival. These findings identify new signaling pathways used by amino acids underscoring the crucial importance of these nutrients in cell metabolism and offering new mechanistic insights in developing pharmaceutically active peptides.

Differential Signaling of Amino Acids to the mTOR Pathway.

Some amino acids are known to control proteogenesis (mTOR kinases) or proteolysis (autophagy) more than others. Recent and older data identify leucine (L), valine (V), isoleucine (I), alanine (A), glutamine (Q), arginine (R), glycine (G), proline (P), and asparagine (N), either alone or in combination, as more potent activators of mTOR or inhibitors of autophagy than other amino acids, such as glutamate (E), threonine (T), serine(S), lysine (K), threonine (T), phenylalanine (F), tyrosine (Y), and methionine (M) that have been reported to have no or opposite effects. Amino acids leucine (L), alanine (A), glutamine (Q), and proline (P) are reported to have most prominent autophagic effects on human cells (AJ Meijer et al Amino Acids 2015, 47, 2037-2063).

Several autophagy inhibiting peptide modulators of mTOR and several organic salts of those peptides, are earlier found useful in treatment of inflammation or addressing issues of hemodynamic instability in PCT/NL2018/052822, PCT/NL2020/050535, PCT/NL2020/050605 or PCT/NL2021/050223. It is disclosed in this application, that autophagy inhibiting peptides of this class have angiogenic properties, as shown in sprouting assays of human vascular endothelial cells, and are useful in healing of diabetic (foot) ulcers in humans.

Several of these peptides carry a binding motif to the human elastin-receptor-complex (ERC); such a binding motif xGxxPx is for example present in a peptide fragment derived from position ˜41-57 in loop 2 of β-human chorionic gonadotrophin (beta-hCG) (see also FIG. 5). Small peptides derived from position ˜41-57 (some specify to 40-54) in β-human chorionic gonadotrophin (beta-hCG) likely protect both mother and child from the maternal immune response during pregnancy (U.S. Pat. Nos. 6,583,109B1, 7,358,330B2, Khan, Immunoregulatory Properties of Break Down Products of Human Choriogonadotropin, thesis, 2010). U.S. Pat. No. 6,583,109B1 identifies LQGVLPALPQVVC as involved, Khan (ibid, 2010) identifies MTRVLQGVLPALPQ as involved, U.S. Pat. No. 7,358,330B2 identifies common motif LOGVLPALPQ (in this present application recognized with core ERC-binding-motif QGVLPA, as at least involved in providing said protection. Khan (ibid, 2010) tests LQGV and VLPALP in type 1 diabetes (T1D):-Both peptides significantly (p<0.0001) delay T1D-onset in NOD mice. Biotempt (U.S. Pat. No. 7,358,330B2) tested LQGV, VLPALP and the respective hCG-derived alanine-replacement peptides in vitro in LPS-studies: LQGV, AQGV, LAGV, LQAV, LOGA, VLPALP, ALPALP, VAPALP, VLAALP, VLPAAP, and VLPALA. All significantly inhibited LPS-activity in vitro. Van den Berg et al., (Synthetic oligopeptides related to the [beta]-subunit of human chorionic gonadotropin attenuate inflammation and liver damage after (trauma) haemorrhagic shock and resuscitation. Shock. 2009 March; 31 (3): 285-91) tests LQGV, AQGV and LAGV in vivo in a rat model of hemorrhagic shock. All three significantly (p<0.001) prevented systemic release of TNF-α and IL-6. Activity of human C-peptide also depends on the xGxxPG motif and is blocked by antagonists of ERC (see also FIG. 6). FIG. 6A shows an upregulating effect of the xGxxPG-bearing human C-peptide on CD4⁺ lymphocyte migration, an important process in early angiogenesis (Kwee et al., CD4 T-cells regulate angiogenesis and myogenesis. Biomaterials. 2018 September; 178:109-121. Also (Lemaire et al. The elastin peptide VGVAPG increases CD4 (+) T-cell IL-4 production in patients with chronic obstructive pulmonary disease. Respir Res. 2021; 22 (1): 14), the prototype xGxxPG binding motif of VGVAPG elastin peptide modulates CD4⁺ lymphocyte IL-4 production indicating similar CD4⁺-binding activities of elastin-derived peptides in angiogenesis. Scrambled C-peptide and pig C-peptide, bearing no xGxxPG-motifs, did not affect CD4⁺-migration. Since Gal-3 is considered expressed by CD4⁺ cells, it was not tested in a similar assay. FIG. 6B shows that the CD4⁺-migratory effects of C-peptide are blunted by V14 peptide and lactose, each being specific inhibitors of xGxxPG-elastin-receptor binding, as demonstrated in the art for peptides with motif VGVAPG. Binding of mid-portion of human C-peptide (-fragments) is thus elastin receptor-specific and depends on the presence of the xGxxPG-motif in angiogenesis, furthermore illustrating that these xGxxPG bearing peptides enter the cell by receptor mediated endocytosis. Indeed, Luppi et al., (Luppi et al., C-peptide is internalised in human endothelial and vascular smooth muscle cells via early endosomes. Diabetologia. 2009 October; 52 (10): 2218-28.) show that C-peptide enters a cell by endocytosis and signals from within the endosomes. According to Luppi et al., (ibid) endosomes represent a signalling station through which C-peptide might achieve its cellular effects. This illustrates the set-up to hydrolysis, a condition necessary for amino acids to activate mTOR and inhibit autophagy. Typically, some amino acids activate mTOR more than others, and therewith inhibit autophagy more than others (see also FIG. 7). Alanine has been shown to have a very specific coregulatory effect on autophagy (Meijer et al., Regulation of autophagy by amino acids and MTOR-dependent signal transduction. Amino Acids. 2015 October; 47 (10): 2037-63; Kadowaki et al. Nutrient control of macroautophagy in mammalian cells. Mol Aspects Med. 2006 October-December; 27 (5-6): 426-43). The combination of alanine and leucine at physiological concentrations elicits the same inhibitory effect as a complete mixture of all 20 amino acids on autophagic proteolysis in rat hepatocytes (Meijer et al, ibid). This same study also showed that the metabolism of alanine was required for its inhibitory effect on autophagy. Also, although the role of L-glutamine (Gln) in nutrition and health have been extensively documented, its effects on the cardiovascular system have just recently come to light (Bertero et al., The molecular rationale for therapeutic targeting of glutamine metabolism in pulmonary hypertension. Expert Opin Ther Targets. 2019 June; 23 (6): 511-524). Tan et al., (Glutamine metabolism regulates autophagy-dependent mTORC1 reactivation during amino acid starvation. Nat Commun. 2017 Aug. 24; 8 (1): 33) show that glutamine metabolism is sufficient to restore mTORC1 activity during prolonged amino acid starvation in an autophagy-dependent manner. Ukai et al (Gtr/Ego-independent TORC1 activation is achieved through a glutamine-sensitive interaction with Pib2 on the vacuolar membrane. PLOS Genet 14 (4): e1007334. https://doi.org/10.1371/journal.pgen.1007334, 2018) show that two molecular machineries, Pib2 and Gtr/Ego, form distinct complexes with TORC1 in a mutually exclusive manner, implying an exclusive functional relationship between TORC1 and Pib2 or Gtr/Rag in response to various amino acids. They also show that the amino acid-dependent activation of TORC1 is achieved through the Pib2 and Gtr/Ego pathways by anchoring them to the vacuolar membrane, and show that glutamine binds directly to the Pib2 complex and enhances Pib2-TORC1 complex formation finding Pib2 as an element of a putative glutamine sensor to mTOR. (see also FIG. 4). Intracellular glutamine also has the capacity to exchange with extracellular essential amino acids https://www.nature.com/articles/ncomms11457-ref-CR14 (Nicklin et al., Bidirectional transport of amino acids regulates mTOR and autophagy. Cell. 2009 Feb. 6; 136 (3): 521-34) and therewith further regulate mTOR activity (Jewell Metabolism. Differential regulation of mTORC1 by leucine and glutamine. Science. 2015 Jan. 9; 347 (6218): 194-8. doi: 10.1126/science.1259472. Epub 2015 Jan. 7). Sun et al (Glycine Regulates Protein Turnover by Activating Protein Kinase B/Mammalian Target of Rapamycin and by Inhibiting MuRF1 and Atrogin-1 Gene Expression in C2C12 Myoblasts. J Nutr. 2016 December; 146 (12): 2461-2467.) find glycine to regulate protein turnover by activating mTORC1 and by inhibiting the expression of genes for proteolysis. Also, glycine-removal by decarboxylase (GLDC, an enzyme belonging to the glycine cleavage system) stimulates autophagy (Zhuang et al., Glycine decarboxylase induces autophagy and is downregulated by miRNA-30d-5p in hepatocellular carcinoma. Cell Death Dis 10, 192 (2019). Furthermore, T2D causes endothelial dysfunction, induces abnormal angiogenesis, and changes serum amino acid metabolism, especially for glycine, which is significantly decreased, due to hyperglycaemia. Metabolomic analysis of amino acid profiles showed that intracellular glycine was significantly lower in induced pluripotent stem cell-derived cells (iPSCs) derived from endothelial cells (EC) from diabetic patients than those cells derived from healthy subjects (Su et al., Diabetic Endothelial Cells Differentiated From Patient iPSCs Show Dysregulated Glycine Homeostasis and Senescence Associated Phenotypes. Front Cell Dev Biol. 2021 May 31; 9:667252), indicating glycine deprivation of EC's in T2D. The branched chain amino acid (BCAA) valine, an essential amino acid for humans, plays a particularly important role in cell growth and metabolism Several studies have highlighted the capacity of valine to increase protein synthesis and milk yield via stimulation of the mammalian target of rapamycin (MTOR) pathway (Long et al., Valine increases milk fat synthesis in mammary gland of gilts through stimulating AKT/MTOR/SREBP1 pathway, Biology of Reproduction, Volume 101, Issue 1, July 2019, Pages 126-137,) In recent years, plasma BCAA concentrations have been considered as novel hallmarks of lipid metabolism homoeostasis. Evidence in human and rat has identified a strong relationship between valine metabolism and fat metabolism. In the control of autophagy, leucine, together with arginine, appears to be the most, or at least a particularly prominent, inhibitory amino acid. In addition, its anti-proteolytic effect was potentiated by insulin and by cell swelling (Meijer et al, ibid). Proline as substrate is stored as collagen in extracellular matrix, connective tissue, and bone and it is rapidly released from this reservoir by the sequential action of matrix metalloproteinases, peptidases, and prolidase. As the only proteinogenic secondary amino acid, proline has special biological effects, serves as a regulator of all protein-protein interactions and responses to metabolic stress, and initiates a variety of downstream metabolic activities, including autophagy (Kadowaki et al. Nutrient control of macroautophagy in mammalian cells. Mol Aspects Med. 2006 October-December; 27 (5-6): 426-43). For one) the enzymes utilizing proline respond to stress signalling; for two) there is a large, mobilizable pool of proline; and for three) the metabolism of proline serves special stress functions. In cancers, proline has been reported to act as a responder to nutrient and oxygen deprivation in autophagy. Moreover, in the last few years, proline has been investigated for its distinctive metabolic functions in neuronal autophagy. It was shown that proline inhibits neuronal autophagy whereas the oxidation of proline by proline-oxidase enhances oxidative stress, that in turn triggers autophagy. Decades of research have established the importance of L-arginine in promoting cardiovascular health through the generation of the gas nitric oxide (NO) by the enzyme NO synthase (NOS). The release of NO by endothelial cells (ECs) regulates blood flow and blood pressure by inhibiting arterial tone. Furthermore, NO maintains blood fluidity and prevents thrombosis by limiting platelet aggregation and adhesion. NO also protects against intimal thickening by blocking smooth muscle cell (SMC) proliferation, migration, and collagen synthesis. Recently, L-asparagine, like glutamine, was found to signal to mTORC1 through Arf1 in the absence of the Rag GTPases, and like glutamine, intracellular asparagine exchanges with extracellular amino acids. Although glutamine can participate in the synthesis of different amino acids through the production of glutamate, only asparagine requires glutamine for de novo synthesis. Asparagine synthetase (ASNS) is the enzyme that converts glutamine and aspartate into glutamate and asparagine in an ATP-dependent manner⁶. ASNS expression is upregulated during amino acid starvation through the activation of activating transcription factor 4 (ATF4). Asparagine has been identified as an exchange amino acid factor that regulates the serine/threonine kinase mammalian target of rapamycin complex 1 (mTORC1), nucleotide biosynthesis and proliferation (Bodineau et al., Two parallel pathways connect glutamine metabolism and mTORC1 activity to regulate glutamoptosis. Nat Commun. 2021 Aug. 10; 12 (1): 4814).

Microvascular angiogenesis (sprouting) assay of human vascular endothelial cells grown in Matrigel (see also FIGS. 8, 9 and 10)

Human pulmonary microvascular endothelial cells (Hpmec, HUVE, or other vascular EC, may also be used) were cultured in EBM-2 medium (Lonza, CC-3156) supplemented with 10% FBS and all components present in the bullet kit (Lonza, CC-4147) containing human recombinant FGF-B, human recombinant VEGF, human recombinant R3-IGF-1, ascorbic acid, human recombinant EGF and GA-1000 (Gentamicin sulfate-Amphotericin). 24 hours prior to the assay Hpmec were starved in EBM-2 medium containing 0.5% FBS instead of 10% FBS. For introduction in the angiogenesis assay, endothelial cells were washed 2 times with warm PBS, trypsinized and resuspended in serum free EBM-2 medium. For the angiogenesis assay, Matrigel (Fisher Scientific, Landsmeer, The Netherlands #11523550) was diluted 1:1 in serum-free EBM-2 medium and plated in a 96-well plate (50 μL). After polymerization, the cells were combined with the experimental peptide fragments and added on top of the Matrigel (100 μL). Pictures of the microvascular network were taken after 24 hours of incubation with a 4× objective and analysed using the plug-in angiogenesis analyser in ImageJ.

### Angiogenesis in Wound-Healing Requires Modulation of Angiopoietin 1 (ANG1) Versus Angiopoietin 2 (ANG2) (See Also FIG. 11).

The angiopoietin family (Ang-1-4) has been shown to play a critical role in the modulation of physiologic angiogenesis and pathologic neovascularization. VEGF and the angiopoietins function together playing independent roles during vascular development and embryogenesis; VEGF acts early during vessel formation and Ang-1 acts later during vessel remodeling, maturation, and stabilization. Angiopoietins 1 and 2 have been studied in in vivo and in vitro models. Ang-1, a well-established secreted 70 KDa ligand that shares many of the pro-angiogenic properties of VEGF, protects blood vessels from increased plasma leakage by counteracting transendothelial permeability stimulated by VEGF. Ang-1 signals primarily through the transmembrane receptor tyrosine kinase (Tie2), which is expressed ubiquitously in vascular endothelium and is phosphorylated in quiescent vessels. Ang-1 interacts with several cells, such as neutrophils, endothelial cells, and fibroblasts, through integrins to mediate survival, cell adhesion, and migration. Ang-1 is an essential and critical regulator of blood vessel development, as evidenced by the Ang-1 null mouse, which is embryonically lethal. In contrast, Ang-2, an antagonist of Ang-1 and Tie2 signalling, is generally not expressed in tissues of healthy adults, but is expressed in secretory tissues undergoing inflammation and vascular remodelling, such as healing wounds.

### Factors Contributing to Decreased Wound Healing (See Also FIG. 12).

Chronic diabetic ulcers are responsible for more than 42,500, non-traumatic lower-limb amputations and 27% of diabetic health care costs in the United States annually. The impairments in the phenotype of cutaneous diabetic wound healing are associated with several intrinsic and extrinsic factors shown. Wounds in diabetic patients, as well as in murine models of Type I and Type II diabetes, show a defect in angiogenesis, re-epithelialization, and wound closure. The initial re-epithelialization does not depend on angiogenesis, but the complete healing and maturation are regulated closely by vascular responses of several cells and cell-matrix interactions involved in angiogenic activities. The deficiencies in angiogenesis have been attributed to poorly managed blood glucose levels and to the related vascular defects in both endothelial cells (EC) and endothelial progenitor cells (EPC). A deficit in neovascularization in diabetes is known to be associated with a compromised response to ischemia in wound healing as a consequence of metabolic derangement, but beyond this vague understanding, the compromised ability to revascularize ischemic tissues in diabetes is poorly understood. A potential mechanism to explain this impairment is a decrease in the expression of angiogenic growth factors, such as vascular endothelial growth factor (VEGF), angiopoietin-1 (Ang-1), and their receptors. Skin samples taken from the peri-wound area from lower extremities (skin within 1 cm from wound margin) of type 2 diabetic subjects has significantly less expression of both VEGF (46%) and Ang-1 (36%) than the skin tissue of nondiabetic subjects. Many groups have shown evidence linking impaired neovascularization with delayed closure of diabetic wounds, and further, that supplementation of angiogenic growth factors, such as VEGF, Ang-1, EGF, bFGF, and HIF1-α, or a combination of these factors via recombinant growth factor therapy or gene transfer, has positive effects on improving neovascularization and outcomes of diabetic wound closure.

### Determination of C-Peptide Deficiencies (See Also FIG. 13).

Panero et al. (Fasting plasma C-peptide and micro- and macrovascular complications in a large clinic-based cohort of type 1 diabetic patients. Diabetes Care. 2009 February; 32 (2): 301-5) assessed residual B-cell function and there with relative C-peptide deficiency by measuring fasting plasma C-peptide (normal values 0.36-1.17 nmol/l; Diagnostics Product Corporation, Los Angeles, CA) in T1D patients with microvascular vasculopathies. They then performed logistic regression analysis in order to study variables independently associated with microvascular (retinopathy, micro- and macroalbuminuria, and diabetic peripheral neuropathy (DPN)) and macrovascular complications (myocardial infarction, angina, coronary artery bypass graft, stroke, and peripheral arteriopathy). The independent role of C-peptide was examined using tertiles of its distribution (<0.06, 0.06-0.10, and 0.11-2.76 nmol/l). With respect to fasting C-peptide values in the lowest tertile (<0.06 nmol/l), higher values were associated with lower prevalence of microvascular complications (odds ratio [OR] 0.59 [95% CI 0.37-0.94]). No association was evident with macrovascular complications (0.77 [0.38-1.58]).

Qiao, et al. (C-peptide is independent associated with diabetic peripheral neuropathy: a community-based study. Diabetol Metab Syndr 9, 12 (2017).) studied the relationship between residual C-peptide and DPN in patients with type 2 diabetes. With increased diabetes duration, the islet function diminishes gradually, resulting in reduced C-peptide and insulin levels and the prevalence of DPN increases. Fasting C-peptide, 2-h postprandial C-peptide and AC-peptide (i.e., 2-h postprandial C-peptide minus the fasting C-peptide) serum concentrations in the non-DPN group were significantly higher than those in the clinical DPN group (all P≤0.040) and the confirmed DPN group (all P<0.002). The three C-peptide parameters were independently associated with DPN (all P<0.05) after adjusting for age, sex, diabetes duration, smoking status, systolic pressure, body mass index, angiotensin-converting enzyme inhibitors/angiotensin receptor blocker use, fasting plasma glucose, HbA1c, triglyceride and estimated glomerular filtration rate. Compared with the AC-peptide quartile 1 (reference), patients in quartile 3 (odds ratio [OR], 0.110; 95% confidence interval [CI] 0.026-0.466; P=0.003) and quartile 4 (OR, 0.012; 95% CI 0.026-0.559; P=0.007) had a lower risk of DPN after adjusting for the confounders. C-peptide was measured by chemiluminescence (Siemens Healthcare Diagnostics, Malvern, USA) on an ADVIA Centaur XP automatic analyzer (Siemens Healthcare Diagnostics) using a sample volume of 200 μL. From these studies it emerges that fasting C-peptide deficiency in patients with DPN is increasingly present at values of below 1 nmol/l, more specifically at values of below 0.5 nmol/l, more specifically at below 0.3 nmol/L, more specifically at values of below 0.3 nmol/l, more specifically at below 0.2 nmol/L, more specifically at values of below 0.1 nmol/l, more specifically at below 0.06 nmol/L. For better differentiation of such a patient, a further analyses may be made by determining post-prandial C-peptide levels (as for example provided by Qiao et al, ibid), indicating that clinically relevant C-peptide deficiencies may already be found post-prandially at below 1 nmol/l. Determinations of AC-peptide (i.e., 2-h postprandial C-peptide minus the fasting C-peptide, as for example provided by Qiao et al, ibid) may further determine whether a patient is suffering from a C-peptide deficiency. In general, patients with of AC-peptide of below 1 nmol/l are deemed at risk for DPN.

Examples of Formulations, Variable after Further Dose-Finding Studies.

### EXAMPLE 1

### QAQGVALQ-Maleate

To prepare 1 L of the composition, mix
QAQGVALQ-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 2

### LQGVLPAL-Maleate

To prepare 1 L of the composition, mix
LQGVLPAL-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 3

### VLQAVLPP-Maleate

To prepare 1 L of the composition, mix
VLQAVLPP-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 4

### PGAYPGQA-Maleate

To prepare 1 L of the composition, mix
PGAYPGQA-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 5

### AQGV-Maleate

To prepare 1 L of the composition, mix
AQGV-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 6

### LQGVL-Maleate

To prepare 1 L of the composition, mix
LQGVL-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 7

### AQGLQ-Maleate

To prepare 1 L of the composition, mix
AQGLQ-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 8

### LQGLQ-Maleate

To prepare 1 L of the composition, mix
LQGLQ-maleate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 9

### QAQGVALQ-Acetate

To prepare 1 L of the composition, mix
QAQGVALQ-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 10

### LQGVLPAL-Acetate

To prepare 1 L of the composition, mix
LQGVLPAL-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 11

### VLQAVLPP-Acetate

To prepare 1 L of the composition, mix
VLQAVLPP-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 12

### PGAYPGQA-Acetate

To prepare 1 L of the composition, mix
PGAYPGQA-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 13

### AQGV-Acetate

To prepare 1 L of the composition, mix
AQGV-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 14

### LQGVL-Acetate

To prepare 1 L of the composition, mix
LQGVL-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 15

### AQGLQ-Acetate

To prepare 1 L of the composition, mix
AQGLQ-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 16

### LQGLQ-Acetate

To prepare 1 L of the composition, mix
LQGLQ-acetate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 17

### QAQGVALQ-Tartrate

To prepare 1 L of the composition, mix
QAQGVALQ-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 18

### LQGVLPAL-Tartrate

To prepare 1 L of the composition, mix
LQGVLPAL-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 19

### VLQAVLPP-Tartrate

To prepare 1 L of the composition, mix
VLQAVLPP-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 20

### PGAYPGQA-Tartrate

To prepare 1 L of the composition, mix
PGAYPGQA-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 21

### AQGV-Tartrate

To prepare 1 L of the composition, mix
AQGV-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 22

### LQGVL-Tartrate

To prepare 1 L of the composition, mix
LQGVL-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 23

### AQGLQ-Tartrate

To prepare 1 L of the composition, mix
AQGLQ-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 24

### LQGLQ-Tartrate

To prepare 1 L of the composition, mix
LQGLQ-tartrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 25

### QAQGVALQ-Citrate

To prepare 1 L of the composition, mix
QAQGVALQ-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 26

### LQGVLPAL-Citrate

To prepare 1 L of the composition, mix
LQGVLPAL-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 27

### VLQAVLPP-Citrate

To prepare 1 L of the composition, mix
VLQAVLPP-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 28

### PGAYPGQA-Citrate

To prepare 1 L of the composition, mix
PGAYPGQA-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 29

### AQGV-Citrate

To prepare 1 L of the composition, mix
AQGV-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 30

### LQGVL-Citrate

To prepare 1 L of the composition, mix
LQGVL-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 31

### AQGLQ-Citrate

To prepare 1 L of the composition, mix
AQGLQ-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 32

### LQGLQ-Citrate

To prepare 1 L of the composition, mix
LQGLQ-citrate—1.8 mol
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 33

QAQGVALQ-Maleate with Short-Acting Insulin

To prepare 1 L of the composition, mix
QAQGVALQ-maleate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 34

LQGVLPAL-Maleate with Intermediate-Acting Insulin

To prepare 1 L of the composition, mix
LOGVLPAL-maleate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 35

VLQAVLPP-Maleate with Rapid-Acting Insulin

To prepare 1 L of the composition, mix
VLQAVLPP-maleate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 36

PGAYPGQA-Maleate with Intermediate-Acting Insulin

To prepare 1 L of the composition, mix
PGAYPGQA-maleate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 37

AQGV-Maleate with Rapid-Acting Insulin

To prepare 1 L of the composition, mix
AQGV-maleate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 38

LQGVL-Maleate with Rapid-Acting Insulin

To prepare 1 L of the composition, mix
LQGVL-maleate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 39

AQGLQ-Maleate with Short-Acting Insulin

To prepare 1 L of the composition, mix
AQGLQ-maleate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 40

LOGLQ-Maleate with Rapid-Acting Insulin

To prepare 1 L of the composition, mix
LQGLQ-maleate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 41

QAQGVALQ-Acetate with Short-Acting Insulin

To prepare 1 L of the composition, mix
QAQGVALQ-acetate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 42

LQGVLPAL-Acetate with Intermediate-Acting Insulin

To prepare 1 L of the composition, mix
LQGVLPAL-acetate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 43

VLQAVLPP-Acetate with Short-Acting Insulin

To prepare 1 L of the composition, mix
VLQAVLPP-acetate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 44

PGAYPGQA-Acetate with Intermediate-Acting Insulin

To prepare 1 L of the composition, mix
PGAYPGQA-acetate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 45

AQGV-Acetate with Rapid-Acting Insulin

To prepare 1 L of the composition, mix
AQGV-acetate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 46

LQGVL-Acetate with Rapid-Acting Insulin

To prepare 1 L of the composition, mix
LQGVL-acetate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 47

AQGLQ-Acetate with Short-Acting Insulin

To prepare 1 L of the composition, mix
AQGLQ-acetate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 48

LQGLQ-Acetate with Rapid-Acting Insulin

To prepare 1 L of the composition, mix
LQGLQ-acetate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 49

QAQGVALQ-Tartrate with Short-Acting Insulin

To prepare 1 L of the composition, mix
QAQGVALQ-tartrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 50

LQGVLPAL-Tartrate with Intermediate-Acting Insulin

To prepare 1 L of the composition, mix
LQGVLPAL-tartrate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 51

VLQAVLPP-Tartrate with Short-Acting Insulin

To prepare 1 L of the composition, mix
VLQAVLPP-tartrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 52

PGAYPGQA-Tartrate with Intermediate-Acting Insulin

To prepare 1 L of the composition, mix
PGAYPGQA-tartrate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 53

AQGV-Tartrate with Rapid-Acting Insulin

To prepare 1 L of the composition, mix
AQGV-tartrate—1.8 mol
Human rapid-acting insulin (2800 U/mg)—100000 U M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 54

LQGVL-Tartrate with Rapid-Acting Insulin

To prepare 1 L of the composition, mix
LQGVL-tartrate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 55

AQGLQ-Tartrate with Rapid-Acting Insulin

To prepare 1 L of the composition, mix
AQGLQ-tartrate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 56

LQGLQ-Tartrate with Short-Acting Insulin

To prepare 1 L of the composition, mix
LOGLQ-tartrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 57

QAQGVALQ-Citrate with Short-Acting Insulin

To prepare 1 L of the composition, mix
QAQGVALQ-citrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 58

LQGVLPAL-Citrate with Short-Acting Insulin

To prepare 1 L of the composition, mix
LQGVLPAL-citrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 59

VLQAVLPP-Citrate with Short-Acting Insulin

To prepare 1 L of the composition, mix
VLQAVLPP-citrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 60

PGAYPGQA-Citrate with Intermediate-Acting Insulin

To prepare 1 L of the composition, mix
PGAYPGQA-citrate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 61

AQGV-Citrate with Rapid-Acting Insulin

To prepare 1 L of the composition, mix
AQGV-citrate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 62

LQGVL-Citrate with Rapid-Acting Insulin

To prepare 1 L of the composition, mix
LQGVL-citrate—1.8 mol
Human rapid-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 63

AQGLQ-Citrate with Short-Acting Insulin

To prepare 1 L of the composition, mix
AQGLQ-citrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 64

LQGLQ-Citrate with Short-Acting Insulin

To prepare 1 L of the composition, mix
LOGLQ-citrate—1.8 mol
Human short-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 65

VGVAPGVGVAPGVGVAPG-Citrate with Intermediate-Acting Insulin

To prepare 1 L of the composition, mix
VGVAPGVGVAPGVGVAPG-citrate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 66

QVGQVELGGGPGAGSLQP-Citrate with Intermediate-Acting Insulin

To prepare 1 L of the composition, mix
QVGQVELGGGPGAGSLQP-citrate—1.8 mol
Human intermediate-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 67

GAYPGAPGAYPGAPAPGV-Citrate with Intermediate-Acting Insulin

To prepare 1 L of the composition, mix
GAYPGAPGAYPGAPAPGV-citrate—1.8 mol
Human intermediate-acting insulin (2800 U/mg)—100000 U

### EXAMPLE 68

VGVAPGVGVAPGVGVAPG-Citrate with Long-Acting Insulin

To prepare 1 L of the composition, mix
VGVAPGVGVAPGVGVAPG-citrate-2.4 mol
Human long-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 69

QVGQVELGGGPGAGSLQP-Citrate with Long-Acting Insulin

To prepare 1 L of the composition, mix
QVGQVELGGGPGAGSLQP-citrate-2.4 mol
Human long-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

### EXAMPLE 70

GAYPGAPGAYPGAPAPGV-Citrate with Long-Acting Insulin

To prepare 1 L of the composition, mix
GAYPGAPGAYPGAPAPGV-citrate-2.4 mol
Human long-acting insulin (2800 U/mg)—100000 U
M-Kreosol—25 mg, Glycerol—160 mg, 0.9% NaCL, optionally water and either 10% hydrochloric acid or 10% sodium hydroxide sufficient to make a composition volume of 1 L and a desired final pH.

What is claimed:

1. An autophagy inhibiting modulator of mTOR for use in angiogenic control comprising inducing angiogenic activity in prevention or treatment of a vasculopathy of a subject, said modulator comprising a source of amino acids, preferably a peptide, wherein said amino acids are for at least 50% selected from the group consisting of alanine (in one letter code: A), glutamine (Q), glycine (G), valine (V), leucine (L), isoleucine (I), proline (P), arginine (R), and asparagine (N).

2. A modulator according to claim 1, for use in prevention or treatment of a nephropathy.

3. A modulator according to claim 1, for use in prevention or treatment of end-stage renal disease.

4. A modulator according to claim 1, for use in prevention or treatment of a retinopathy.

5. A modulator according to claim 1, for use in prevention or treatment of blindness.

6. A modulator according to claim 1, for use in prevention or treatment of a neuropathy.

7. A modulator according to claim 1, for use in prevention or treatment of a peripheral diabetic neuropathy

8. A modulator according to claim 1, for use in prevention or treatment of a diabetic ulcer.

9. A modulator according to claim 1, wherein said subject is also treated to achieve or maintain glycaemic control.

10. A modulator according to claim 9, wherein said subject is also treated with an insulin to achieve or maintain glycaemic control.

11. A modulator according to claim 1, wherein said vasculopathy comprises a diabetic vasculopathy.

12. A modulator according to claim 1, wherein said subject is having or suspected of having a C-peptide deficiency.

13. A formulation of a growth factor and an autophagy inhibiting modulator of mTOR, said modulator comprising a source of amino acids, preferably a peptide, wherein said amino acids are for at least 50% selected from the group consisting of alanine (in one letter code: A), glutamine (Q), glycine (G), valine (V), leucine (L), isoleucine (I), proline (P), arginine (R), and asparagine (N).

14. A formulation according to claim 13, wherein said growth factor comprises an insulin for use in glycaemic control as well as an autophagy inhibiting modulator of mTOR for use in angiogenic control.

15. A pharmaceutical formulation comprising a formulation according to claim 13.

16. A method for inducing angiogenic activity in the prevention or treatment of a vasculopathy of a subject, comprising administering to the subject a formulation according to claim 13.

17. The method according to claim 16, wherein the method comprises prevention or treatment of a nephropathy.

18. The method according to claim 16, wherein the method comprises prevention or treatment of end-stage renal disease.

19. The method according to claim 16, wherein the method comprises prevention or treatment of a retinopathy.

20. The method according to claim 16, wherein the method comprises prevention or treatment of blindness.

21. The method according to claim 16, wherein the method comprises prevention or treatment of a neuropathy.

22. The method according to claim 16, wherein the method comprises prevention or treatment of a peripheral diabetic neuropathy

23. The method according to claim 16, wherein the method comprises prevention or treatment of an diabetic ulcer.

24. The method according to claim 16, wherein said vasculopathy comprises a diabetic vasculopathy.

25. The method according to claim 16, wherein said subject is having or suspected of having a C-peptide deficiency.

26. A method for inducing angiogenic activity in the prevention or treatment of a vasculopathy of a subject, comprising administering to the subject an autophagy inhibiting modulator of mTOR, said modulator comprising a source of amino acids, preferably a peptide, wherein said amino acids are for at least 50% selected from the group consisting of alanine (in one letter code: A), glutamine (Q), glycine (G), valine (V), leucine (L), isoleucine (I), proline (P), arginine (R), and asparagine (N).
